(19) United States
(12) Patent Application Publication
(10) Pub. No.: US 2025/0139335 A1
(43) Pub. Date: May 1, 2025

(54) SYSTEMS AND METHODS FOR THE IDENTIFICATION OF TARGET-SPECIFIC T CELLS AND THEIR RECEPTOR SEQUENCES USING MACHINE LEARNING

(71) Applicant: IMMUNOSCAPE PTE LTD, Singapore (SG)

(72) Inventors: Andreas WILM, Singapore (SG); Loan Ping ENG, Singapore (SG); Florian SCHMIDT, Singapore (SG)

(21) Appl. No.: 18/690,702

(22) PCT Filed: Sep. 7, 2022
(86) PCT No.: PCT/IB2022/000512

§ 371 Date: Mar. 8, 2024

(30) Foreign Application Priority Data
Sep. 10, 2021 (SG) 10202109992T
Apr. 28, 2022 (SG) 10202204588Y
Jun. 15, 2022 (SG) 10202250170C

(51) Int. Cl. G06F 30/27; G16B 15/30; G16B 25/00; G16B 30/00; G16B 40/20

(52) U.S. Cl. CPC G06F 30/27; G16B 15/30; G16B 25/00; G16B 30/00; G16B 40/20

(57) ABSTRACT

The present application describes a computer-implemented method for identifying target-specific T cells and their T cell Receptor (TCR) sequences. The method includes deriving single cell T cell data from a sample. The data comprises T cell profile and T cell TCR sequence. The method also includes selecting candidate T cells and their TCR sequences from the single cell T cell data using a machine learning classifier that is trained to classify T cells based on their profiles. The method may also include aggregating results over clonotypes, adding T cells with similar TCR sequences and ranking the list of candidates.

### TECHNICAL FIELD

The disclosed implementations relate generally to diagnosis and treatment of diseases, and more specifically to identification of target-specific T cells and their receptor sequences using machine learning.

### BACKGROUND

Killer T cells are part of the human adaptive immune response that defends against foreign invaders. These cells kill diseased cells (e.g., cancer cells, virus infected cells) by first binding to them. Binding is facilitated through a T cell receptor (TCR), which is unique per T cell and whose sequence determines its specificity. TCRs bind to disease-specific antigens presented as peptides in the context of surface major histocompatibility complex (MHC) or HLA molecules, such as a viral-derived or a tumor-derived peptide. Knowledge of disease-specific TCR sequences would allow their use in adoptive TCR-T cell therapy or other therapeutic strategies for cancer or infectious diseases. The TCR sequences can also be used to monitor T cells of interest in a patient during disease or treatment, or to diagnose a patient with a disease, such as cancer, autoimmune or infectious disease.

The human body hosts a vast number of TCR sequences (estimated to exceed 1010), and the space of the potential peptide antigens recognized by these TCRs is even bigger. Thus, finding the TCR sequence(s) of interest is extremely difficult. This can be partly addressed by searching for antigen-specific T cells against a panel of predicted or known antigens (e.g., using peptide-MHC multimers as probes for antigen-specific T cells). But antigen prediction algorithms are not perfect and only a limited number of antigens (e.g., a few hundred) can be tested empirically for T cell recognition.

### SUMMARY

Accordingly, there is a need for methods that identify T cells and their TCR sequences that are specific for a certain disease from multiomics datasets. Some implementations use healthy and disease reference T cell datasets, produced with different types of wet-lab technologies based on deriving high-dimensional single cell data from the T cells. Using these technologies, some implementations derive multiple sets of T cell information, at the single cell level: antigen specificity, cell phenotype (protein marker expression and/or gene expression), and TCR sequence. These technologies are sometimes called TargetScape and TCR Antigen Profiling (TAP), but any single cell technology based on mass cytometry, flow cytometry, single cell sequencing, spatial transcriptomics, spatial proteomics or other similar platforms, and capable of generating these sets of high-dimensional T cell data, could be used.

Some implementations screen T cells against a multiplexed peptide-MHC multimer antigen panel together with an antibody panel (e.g., TargetScape, where the read-out is performed by mass cytometry). Some implementations screen T cells against peptide-MHC multimers together with an antibody panel and together with gene and TCR sequencing (e.g., TAP, where the read-out is performed by single cell sequencing). These panels and other T cell profiling are used to infer the phenotypes of T cells recognizing specific antigens. Using these T cell data, some implementations train machine learning (ML) classifiers that can classify T cells of interest for which there is no actual knowledge of the antigen target. T cells recognizing a certain antigen specific for a certain disease or virus are likely to have common characteristics, such as phenotypic protein marker combinations, gene expression patterns and TCR sequence similarity. ML models can be trained to learn such characteristics and can then later be used to predict the target specificity from the T cell profile, which may include protein marker expression patterns, gene expression patterns, and/or TCR sequences.

In one aspect, a method is provided for identifying target-specific T cells. The method includes deriving single cell T cell data from a sample, wherein the data comprises T cell profiles. In some implementations, phenotypic protein marker T cells profiles are obtained by screening T cells with an antibody panel (e.g., TargetScape, where the read-out is performed by mass cytometry). In some implementations, phenotypic protein marker and/or gene expression T cell profiles are derived by screening T cells with an antibody panel together with gene and TCR sequencing (e.g., TAP, where the read-out is performed by single cell sequencing). The method also includes forming feature vectors which may involve normalizing and rescaling the T cell profile, depending on the nature of the considered features. The method also includes selecting candidate T cells from the single cell T cell data by inputting the feature vectors to a machine learning classifier that is trained to classify T cells based on their profiles. Some implementations use an ML classifier that was trained on phenotypic protein markers to predict cells of interest. Some implementations use an ML classifier that was trained on gene expression profiles to predict cells of interest. Some implementations use an ML classifier that was trained on a combination of phenotypic protein markers and gene expression profiles and/or TCR sequence features to predict cells of interest. Some implementations aggregate ML predictions over groups of cells with identical TCR sequence composition, so called clonotypes. Some implementations then rank the resulting list of candidate TCRs. Some implementations also identify target-specific T cells based on TCRs sequences similar to the TCR known to bind and/or predicted by ML to bind to a given target. TCR sequences are protein sequences made up from two chains (alpha and beta), each made up of multiple segments (called V, J and optionally D). The intersection of these segments are termed Complementarity-Determining Region (CDR). CDR3A and CDR3B within, respectively, the TCRalpha and TCRbeta chains, are the TCR domains with highest diversity and are primarily responsible for binding to the target peptide-MHC complex. Similar TCR sequences may include TCRs with CDR3A and/or CDR3B amino acid sequences that are similar, i.e. typically differ by up to two amino acids. Similar TCR sequences may also include TCRs with CDR3A and/or CDR3B with similar physicochemical characteristics. This classification process identifies putative disease-specific TCR sequences recognizing tumor targets, viral targets, or other antigens of interest, for application in disease diagnosis and/or immune-monitoring. The common characteristics of putative disease-specific T cells can also be used to monitor disease-specific T cells in patients during disease or treatment. In some implementations, ML-predicted TCR sequences recognizing targets of interest encode for isolated polypeptides that can be expressed in host cells, for example T cells, to direct the T cells towards the target. In some implementations, the TCR polypeptide is expressed by transducing the host cells with a vector, for example a lentiviral vector, encoding for an isolated nucleic acid coding for the TCR sequence. In some implementations, the TCR-expressing host cells and/or the vector are part of a pharmaceutical formulation comprising a pharmaceutically acceptable carrier.

In another aspect, a method is provided for training a machine learning classifier for identifying target-specific T cells. The method includes generating reference datasets for a healthy cohort data and a disease cohort data using one or more techniques to screen T cells for antigen specificity, measure cell-associated protein levels and/or gene expression, and derive TCR sequences. The method also includes training one or more machine learning classifiers to classify target-specific T cells based on their profiles using the reference datasets.

In another aspect, a system configured to perform any of the above methods is provided, according to some implementations.

### BRIEF DESCRIPTION OF THE DRAWINGS

For a better understanding of the various described implementations, reference should be made to the Description of Implementations below, in conjunction with the following drawings in which like reference numerals refer to corresponding parts throughout the figures.

FIG. 1 shows a T cell that recognizes the antigen peptide on a tumor or infected cell by binding to it with its T cell receptor (TCR).

FIG. 2 shows a schematic diagram of an assay workflow for a single sample using a mass cytometry-based method (sometimes referred to as TargetScape) followed by TCR Antigen Profiling (sometimes referred to as TAP; a single cell sequencing based method), according to some implementations.

FIG. 3 shows a schematic diagram of generated reference datasets, according to some implementations.

FIG. 4 shows a flowchart of reference datasets generation and machine learning model training.

FIG. 5 shows a flowchart of an example method for identifying target-specific T cells and their TCR sequences, according to some implementations.

FIG. 6 shows a schematic overview of the machine learning based training and predictions of target specificity for T cells.

FIG. 7A shows a flowchart of an example implementation for identifying target-specific T cells from TAP data using models trained on Targetscape data, according to some implementations. FIG. 7B shows example training classification results for TargetScape data for each binary random forest model, while FIG. 7C shows example classification results for a validation dataset from TAP using the ensemble classifier. FIG. 7D shows examples of two clonotypes with target predictions (one viral and one cancer) aggregated over all cells constituting the clonotype. FIG. 7E shows the T cell signatures for each of the six random forest binary classifiers of an example implementation for identifying target-specific T cells from TAP data using models trained on Targetscape data, based on feature importance.

FIG. 8A shows a flowchart of an example implementation for identifying target-specific T cells using T cell profiles from single cell sequencing-based data. FIG. 8B illustrates the composition of T cell profiles. FIG. 8C shows example classification results for a validation dataset using a multi class logistic regression classifier, using gene expression data to generate T cell profiles. FIG. 8D shows example classification results for a validation dataset using a multi class logistic regression classifier, using protein markers (ADT) to generate T cell profiles. FIG. 8E shows example classification results for a validation dataset using a multi class logistic regression classifier, using both gene expression data and protein markers to generate T cell profiles. FIG. 8F shows examples for two clonotypes, predicted as specific for EBV (64 cells) and tumor-associated antigen (TAA) (25 cells) respectively, as well as the top five features inferred by the model for each target specificity class.

FIG. 9A shows a graph plot for results of a functional validation of predicted target specificity for a TCR (named A0015), expressed into a T cell line using a vector.

FIG. 9B shows a graph plot for results of a functional validation of predicted target specificity for a second TCR (named A0099), expressed into a T cell line using a vector.

FIG. 10 shows an example of an EBV-specific clonotype network based on CDR3A and CDR3B sequence similarity.

FIG. 11 shows an example of a Flu-specific network based on similarity in the physicochemical properties of CDR3A and CDR3B amino acid sequences for the shown clonotypes.

### DETAILED DESCRIPTION

### I. Definitions

The practice of the present invention will employ, unless otherwise indicated, conventional techniques of computational biology, machine learning, cell biology, cell culture, molecular biology, transgenic biology, microbiology, recombinant DNA, and immunology, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press); Oligonucleotide Synthesis (M. J. Gait ed., 1984; Mullis et al. U.S. Pat. No. 4,683,195); Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986).

In order that the present disclosure can be more readily understood, certain terms are first defined. As used in this application, except as otherwise expressly provided herein, each of the following terms shall have the meaning set forth below. Additional definitions are set forth throughout the application.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

It is understood that wherever aspects are described herein with the language “comprising,” otherwise analogous aspects described in terms of “consisting of” and/or “consisting essentially of” are also provided.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “consisting essentially of” refer to a value or composition that is within an acceptable error range for the particular value or composition as determined by one of ordinary skill in the art, which will depend in part on how the value or composition is measured or determined, i.e., the limitations of the measurement system. For example, in some embodiments, “about” or “consisting essentially of” can mean within 1 or more than 1 standard deviation per the practice in the art. Alternatively, “about” or “consisting essentially of” can mean a range of up to 10% (i.e., +−10%).

The term “T cell receptor” (TCR), as used herein, refers to a heteromeric cell-surface receptor capable of specifically interacting with a target antigen. As used herein, “TCR” includes but is not limited to naturally occurring and non-naturally occurring TCRs; full-length TCRs and antigen binding portions thereof, chimeric TCRs; TCR fusion constructs; and synthetic TCRs. In humans, TCRs are expressed on the surface of T cells, and they are responsible for T cell recognition and targeting of antigen presenting cells. Target cells display fragments of foreign or self-proteins (antigens) complexed with the major histocompatibility complex (MHC; also referred to herein as complexed with an HLA molecule, e.g., an HLA class 1 molecule). A TCR recognizes and binds to the antigen: HLA complex and recruits CD3 (expressed by T cells), activating the TCR. The activated TCR initiates downstream signaling and an immune response, including the destruction of the target cell.

In general, a TCR can comprise two chains, an alpha chain and a beta chain (or less commonly a gamma chain and a delta chain), interconnected by disulfide bonds. Each chain comprises a variable domain (alpha chain variable domain and beta chain variable domain) and a constant region (alpha chain constant region and beta chain constant region). The variable domain is located distal to the cell membrane, and the variable domain interacts with an antigen. The constant region is located proximal to the cell membrane. A TCR can further comprise a transmembrane region and a short cytoplasmic tail. As used herein, the term “constant region” encompasses the transmembrane region and the cytoplasmic tail, when present, as well as the traditional “constant region.”

The variable domains can be further subdivided into regions of hypervariability, termed complementarity determining regions (CDRs), interspersed with regions that are more conserved, termed framework regions (FR). Each alpha chain variable domain and beta chain variable domain comprises three CDRs and four FRs: FR1, CDR1, FR2, CDR2, FR3, CDR3, FR4. Each variable domain contains a binding domain that interacts with an antigen. Though all three CDRs on each chain are involved in antigen binding, CDR3 is believed to be the primary antigen binding region. CDR1 and CDR2 are believed to primarily recognize the HLA complex.

Where not expressly stated, and unless the context indicates otherwise, the term “TCR” also includes an antigen-binding fragment or an antigen-binding portion of any TCR disclosed herein, and includes a monovalent and a divalent fragment or portion, and a single chain TCR. The term “TCR” is not limited to naturally occurring TCRs bound to the surface of a T cell. As used herein, the term “TCR” further refers to a TCR described herein that is expressed on the surface of a cell other than a T cell (e.g., a cell that naturally expresses or that is modified to express CD3, as described herein), or a TCR described herein that is free from a cell membrane (e.g., an isolated TCR or a soluble TCR).

An “antigen binding molecule,” “portion of a TCR,” or “TCR fragment” refers to any portion of a TCR less than the whole. An antigen binding molecule can include the antigenic complementarity determining regions (CDRs).

An “antigen” refers to any molecule, e.g., a peptide, that provokes an immune response or is capable of being bound by a TCR. An “epitope,” as used herein, refers to a portion of a polypeptide that provokes an immune response or is capable of being bound by a TCR. The immune response may involve either antibody production, or the activation of specific immunologically competent cells, or both. A person of skill in the art would readily understand that any macromolecule, including virtually all proteins or peptides, can serve as an antigen. An antigen and/or an epitope can be endogenously expressed, i.e. expressed by genomic DNA, or can be recombinantly expressed. An antigen and/or epitope can be of exogenous origin. An antigen and/or epitope can possess modifications to the amino acids comprising the antigen and/or epitope if of polypeptide origin (e.g. phosphorylation, glycosylation, cysteinylation, deamidation, and/or other post-translational modifications to the amino acids within the antigen and/or epitope). An antigen and/or an epitope can be specific to a certain tissue, such as a cancer cell, or it can be broadly expressed. In addition, fragments of larger molecules can act as antigens. In one embodiment, antigens are tumor antigens. An epitope can be present in a longer polypeptide (e.g., in a protein), or an epitope can be present as a fragment of a longer polypeptide. In some embodiments, an epitope is complexed with a major histocompatibility complex (MHC; also referred to herein as an HLA molecule, e.g., an HLA class 1 molecule).

“Antigen-derived”, for example “EBV-derived”, refers to an immunogenic peptide/epitope being a portion of the antigen/polypeptide from which it has been processed. For example, an antigen is processed in the cell by the proteasome or immunoproteasome and the resulting antigen-derived peptides are presented on the MHC class I or MHC class II complex.

A “cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” can include a tumor.

An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including Abs, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

A “patient” as used herein includes any human who is afflicted with a cancer (e.g., a lymphoma or a leukemia, or a solid tumor). The terms “subject” and “patient” are used interchangeably herein.

The term “HLA,” as used herein, refers to the human leukocyte antigen. HLA genes encode the major histocompatibility complex (MHC) proteins in humans. MHC proteins are expressed on the surface of cells and are involved in activation of the immune response. HLA class I genes encode MHC class I molecules, which are expressed on the surface of cells in complex with peptide fragments (antigens) of self or non-self proteins. T cells expressing TCR and CD3 recognize the antigen: MHC class I complex and initiate an immune response to target and destroy antigen presenting cells displaying non-self proteins.

As used herein, an “HLA class I molecule” or “MHC class I molecule” refers to a protein product of a wild-type or variant HLA class I gene encoding an MHC class I molecule. Accordingly, “HLA class I molecule” and “MHC class I molecule” are used interchangeably herein.

The MHC Class I molecule comprises two protein chains: the alpha chain and the B2-microglobulin (§ 2m) chain. Human β 2m is encoded by the B2M gene. The alpha chain of the MHC Class I molecule is encoded by the HLA gene complex. The HLA complex is located within the 6p21.3 region on the short arm of human chromosome 6 and contains more than 220 genes of diverse function. The HLA gene are highly variant, with over 20,000 HLA alleles and related alleles, including over 15,000 HLA Class I alleles, known in the art, encoding thousands of HLA proteins, including over 10,000 HLA Class I proteins (see, e.g., hla.alleles.org, last visited Feb. 27, 2019). There are at least three genes in the HLA complex that encode an MHC Class I alpha chain protein: HLA-A, HLA-B, and HLA-C. In addition, HLA-E, HLA-F, and HLA-G encode proteins that associate with the MHC Class I molecule.

As used herein, the term “nucleic acid” refers to a polymer comprising multiple nucleotide monomers (e.g., ribonucleotide monomers or deoxyribonucleotide monomers). “Nucleic acid” includes, for example, genomic DNA, cDNA, RNA, and DNA-RNA hybrid molecules. Nucleic acid molecules can be naturally occurring, recombinant, or synthetic. In addition, nucleic acid molecules can be single-stranded, double-stranded or triple-stranded. In some embodiments, nucleic acid molecules can be modified. In the case of a double-stranded polymer, “nucleic acid” can refer to either or both strands of the molecule.

The term “nucleotide sequence,” in reference to a nucleic acid, refers to a contiguous series of nucleotides that are joined by covalent linkages, such as phosphorus linkages (e.g., phosphodiester, alkyl and aryl-phosphonate, phosphorothioate, phosphotriester bonds), and/or non-phosphorus linkages (e.g., peptide and/or sulfamate bonds). In certain embodiments, the nucleotide sequence encoding, e.g., a target-binding molecule linked to a localizing domain is a heterologous sequence (e.g., a gene that is of a different species or cell type origin).

The terms “nucleotide” and “nucleotide monomer” refer to naturally occurring ribonucleotide or deoxyribonucleotide monomers, as well as non-naturally occurring derivatives and analogs thereof. Accordingly, nucleotides can include, for example, nucleotides comprising naturally occurring bases (e.g., adenosine, thymidine, guanosine, cytidine, uridine, inosine, deoxyadenosine, deoxythymidine, deoxyguanosine, or deoxycytidine) and nucleotides comprising modified bases known in the art.

As will be appreciated by those of skill in the art, in some aspects, the nucleic acid further comprises a plasmid sequence. The plasmid sequence can include, for example, one or more operatively linked sequences selected from the group consisting of a promoter sequence, a selection marker sequence, and a locus-targeting sequence.

The term “sequence identity” means that two nucleotide or amino acid sequences, when optimally aligned, such as by the programs GAP or BESTFIT using default gap weights, share at least, e.g., at least about 70% sequence identity, at least about 80% sequence identity, at least about 85% sequence identity, at least about 90% sequence identity, at least 95% sequence identity, at least about 99% sequence identity, or more. For sequence comparison, typically one sequence acts as a reference sequence (e.g., parent sequence), to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al. 2000, Current Protocols in Molecular Biology). One example of an algorithm that is suitable for determining percent sequence identity and sequence similarity is the BLAST algorithm, which is described in Altschul et al, J. Mol. Biol. 215:403 (1990). Software for performing BLAST analyses is publicly available through the National Center for Biotechnology Information (publicly accessible through the National Institutes of Health NCBI internet server). Typically, default program parameters can be used to perform the sequence comparison, although customized parameters can also be used. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89:10915 (1989)).

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

The term “isolated” refers to a composition, compound, substance, or molecule altered by the hand of man from the natural state. For example, a composition or substance that occurs in nature is isolated if it has been changed or removed from its original environment, or both. For example, a polynucleotide or a polypeptide naturally present in a living animal is not isolated, but the same polynucleotide or polypeptide separated from the coexisting materials of its natural state is isolated, as the term is employed herein.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some versions contain an intron(s).

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof. An exemplary “peptide” of a length of normally between 8 and 12 amino acids, presented on MHC-I, represents the molecular structure recognized by a TCR. A “peptide” can be interchangeably called a “T cell epitope” or “epitope”.

The phrase “antigenic specificity,” as used herein, means that the TCR can specifically bind to and immunologically recognize an antigen. Exemplary antigens include, but are not limited to EBV antigens, CMV antigens, influenza virus antigens, SARS-COV2 antigens, and tumor-associated antigens.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

By the term “specifically binds,” as used herein with respect to a T cell receptor, is meant a T cell receptor which recognizes a specific antigen complexed with an MHC molecule, but does not substantially recognize or bind other antigen: MHC complexes in a sample.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein, the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

In various embodiments, the invention includes one or more of the features defined hereinabove.

### II. Description of Implementations

Reference will now be made in detail to implementations, examples of which are illustrated in the accompanying drawings. In the following detailed description, numerous specific details are set forth in order to provide a thorough understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

It will also be understood that, although the terms first, second, etc. are, in some instances, used herein to describe various elements, these elements should not be limited by these terms. These terms are only used to distinguish one element from another. For example, a first electronic device could be termed a second electronic device, and, similarly, a second electronic device could be termed a first electronic device, without departing from the scope of the various described implementations. The first electronic device and the second electronic device are both electronic devices, but they are not necessarily the same electronic device.

### Protocols to Derive T Cell Profiles

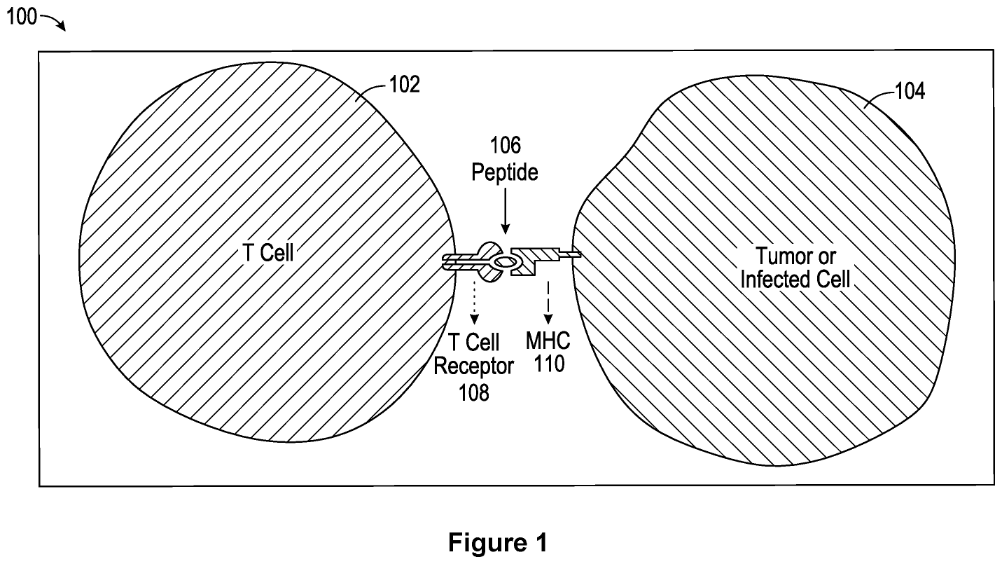

FIG. 1 shows a schematic diagram of a process 100 by which a T cell 102 recognizes an antigen peptide 106 on a tumor or an infected cell 104 by binding to it with its T cell receptor (TCR) 108. As described in the Background section, TCRs 108 bind to disease-specific antigens presented as peptides, such as a viral-derived or a tumor-derived peptide, in the context of surface major histocompatibility cells (MHC) molecules 110.

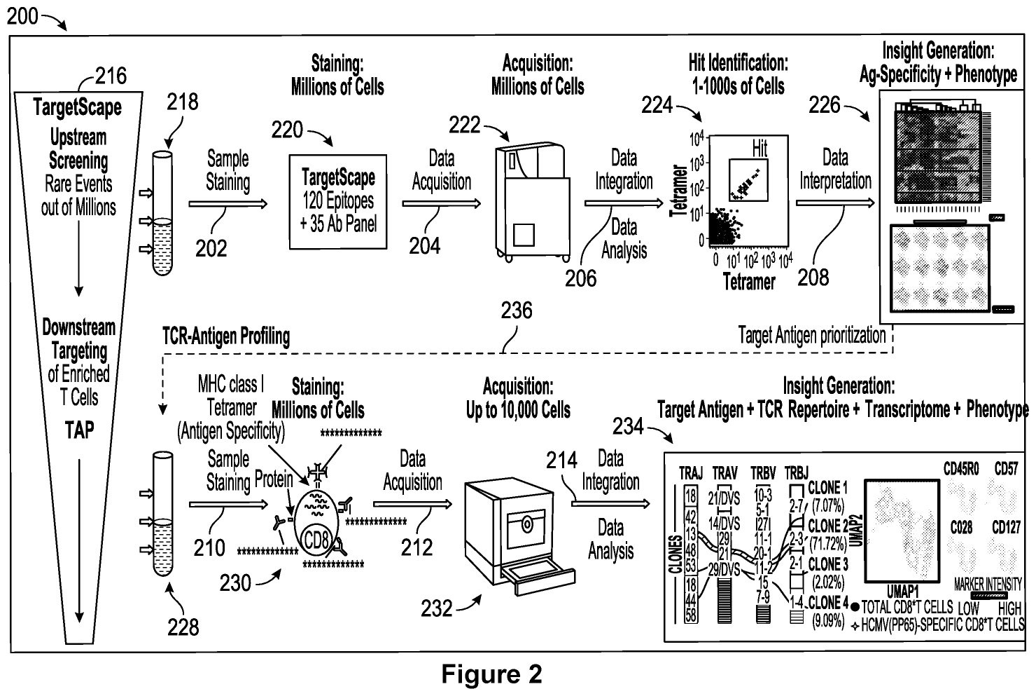

FIG. 2 shows a schematic diagram of a typical assay workflow 200 to acquire data for the reference datasets that are used for training machine learning classifiers, according to some implementations. Two aliquots 218 and 228 of peripheral blood immune cells (PBMC) from a same donor are subjected to a two-step process 216. First, aliquot 218 is subjected to a (mass cytometry-based) TargetScape-based screen where tens of thousands to millions of cells are screened for reactivity against up to hundreds of antigens. This screening process can detect rare TCR/peptide binding events (down to the order of one out of a million cells). TargetScape screens for T cell antigen reactivity and also measures T cell phenotypes based on cell-associated markers. The method multiplexes up to several hundred different peptide-MHC multimers (tetramers) by giving each different peptide-MHC an unique metal or fluorochrome barcode. The cells in the sample are stained with all the barcoded tetramers and a panel of metal-labeled or fluorochrome-labeled antibodies against the cell-associated markers 220. Antibodies typically include commercial antibodies that recognize immune cell protein markers, and in particular T cell markers, such as for example CD3, CD8, CD45RO, KLRG1, CD27, CD38, CD28, PD-1, and any other markers well known in the field that may be useful for describing the T cell profile. Thus, phenotype information is obtained for each and all antigen-specific T cells identified. The cells are then acquired in a mass cytometry or a flow cytometry instrument 222 for detection of cell-associated metals or fluorochromes, respectively. Acquired data is subsequently subjected to data integration and data analysis (208) to identify T cell antigen targets or hits. The analysis output consists of antigen specificity and phenotypic marker values per T cell. This method has been published in scientific journals, see e.g. Fehlings et al. Journal for ImmunoTherapy of Cancer (2019) 7:249, Kared et al. J Clin Invest. 2021; 131(5):e145476 and Newell et al. Nature Methods 2009; 6:497.

The information derived from TargetScape is used to prioritize antigens targets for T cell screening in the subsequent TAP assay, which is single cell sequencing-based. For TAP, aliquot 228 is subjected to staining with oligonucleotide-tagged peptide-MHC class I multimers (210), oligonucleotide-tagged antibodies (for example, CITE-Seq or other equivalent methods) and appropriate barcodes (230). In some instances, T cells of interest may be enriched or sorted before or after the staining. Cells are then processed to obtain barcoded single cells with methods known in the art; for example, this could be done with the 10× Genomics platform, where each single cell is oligonucleotide-barcoded, or with single cell sorting, where each single cell is sorted into a separate well for analysis, or with other platforms enabling downstream single cell sequencing. Libraries are then prepared and appropriately sequenced to obtain RNA sequencing information, TCR sequence information, T cell phenotype and antigen-specificity from the bound peptide-MHC multimers and antibodies. Thus cell-associated protein marker expression, gene expression, antigen specificity, and TCR sequences are derived for thousands of cells (232). The data 232 are subjected to integration and analysis (214) to generate insights on target antigens, TCR repertoire, transcriptome, and/or phenotypes (234). The analysis result is basically an extended version of the TargetScape output tables, i.e. antigen specificity and phenotypic marker values, with many more columns (more phenotypic markers, added TCR sequence and gene expression in addition to antigen specificity).

In some implementations, the assay workflow to acquire data for the reference datasets that are used for training the machine learning classifiers may include only TargetScape-derived data, or only TAP-derived data, or similar types of single cell data derived with methods based on mass cytometry, flow cytometry, single cell sequencing, spatial transcriptomics, spatial proteomics or other similar platforms.

In some implementations, the TAP-derived data may include cell-associated expression markers only, or gene expression only, to derive T cell phenotype information, in addition to the TCR sequences.

### Protocols to Derive TCR Similarities and Clonotype Networks

Sequence based metrics to derive TCR similarities: The similarity of two TCR sequences can be measured by comparing the sequence composition of their CDR3A and CDR3B domains, e.g. assessing the amino acid differences between the two CDR3A sequences and/or the two CDR3B sequences of the two TCRs. Different scores can be assigned to the type of difference in sequence, e.g. substitutions, insertions or deletions. These scores can further be weighted depending on the type of substitution or position within the sequence. One such distance measure is implemented in TCRdist3 (Mayer-Blackwell et al., 2021).

Physicochemical based properties to derive TCR similarities: TCR sequences can also be described in terms of physicochemical and structural properties of its amino acids. Examples of these amino acid properties include hydrophobicity, charge, polarity, polarizability, normalized van der Waals volume and solvent accessibility. For each property, the twenty standard amino acids can be assigned into different groups based on their attributes (see e.g. Dubchak et al. 1995, Dubchak et al. 1999, Li et al. 2006, Cui et al. 2007). For example, certain amino acids are positively charged while others are negatively charged. In another example, amino acids tend to be either buried, intermediate or exposed in the secondary structure, thus describing the solvent accessibility. For some properties such as polarity and normalized van der Waals volume, groups are decided based on the range of values measured for the amino acid. These property groups based on the amino acids can then be used to locally and globally describe the TCR sequence using three descriptors: composition, transition and distribution (see e.g. Dubchak et al. 1995, Dubchak et al. 1999). Composition describes the percentage frequency of a particular amino acid property group in the sequence. Transition describes the percentage frequency of amino acid of a particular property group followed by amino acid of a differing property. Distribution calculates the fractions of the entire peptide sequence where amino acid of a particular property is located within the sequence. TCR sequences can then be represented by values calculated using these descriptors of different amino acid physicochemical properties. These representations can then be converted into a pairwise distance/similarity measure, by for example applying correlation measures, euclidean or manhattan distance or cosine similarity.

Network analysis to derive TCR similarities: To derive groups of similar TCRs, network analysis can be used. Input for this network is a global distance/similarity matrix constructed from all pairwise distance/similarity measures (see above) computed for all possible TCR pairs. Groups of connected sequences can then be found by converting the distance matrix into a graph. This is achieved by representing TCRs as nodes and creating an edge to other nodes/TCRs if the respective distance is below a given threshold.

### Example Reference Data Sets

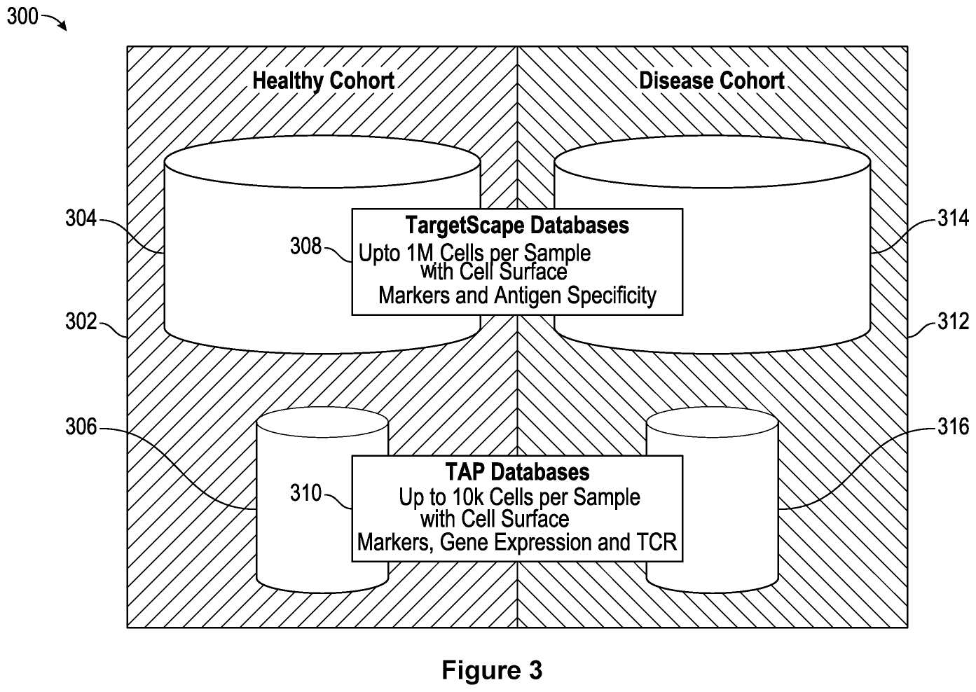

FIG. 3 shows a schematic diagram of the reference datasets 300 generated for healthy and disease cohorts, according to some implementations. In the example shown, four multiomics reference data sets 304, 306, 314, and 316 are created by applying two wet-lab technologies (TargetScape and TAP) to a healthy cohort and the disease cohort respectively. Each dataset is created by analyzing each sample first with TargetScape, screening hundreds of antigens and up to millions of cells (generating 304 & 314). This is followed by TAP-based screening, where antigen hits found in TargetScape are reused to screen T cells and more detailed data is generated for a smaller set of T cells (generating 306 & 316). TargetScape databases 308 generally contain tens or hundreds of thousands of cells per sample with measured cell-associated protein markers and antigen specificity. TAP databases 310 generally include several thousands of cells per sample with measured cell-associated protein markers, antigen specificity, gene expression, and TCR sequence.

### Example TargetScape Databases

The first two data sets 304 and 314 are created by screening blood-derived CD8 T cells with the wet-lab technology called TargetScape. TargetScape is a mass cytometry based technology, which allows to simultaneously screen millions of T cells for recognition of hundreds of peptide-MHC antigens while also measuring the T cell-associated protein markers using specific antibodies, at the single cell level. Some implementations create one database of T cell characteristics (i.e., antigen specificity and protein markers) for each cohort (i.e., a healthy cohort and a disease cohort).

In some implementations, TargetScape measures, via cytometry by time of flight (CyTOF), each T cell antigen peptide specificity and protein markers for a few dozen of protein markers, which is collectively described as cell (protein) phenotype. TargetScape databases 308 are conceptually a table or dataframe that contains protein marker intensities, antigen target and sample identifier per cell. An example TargetScape dataframe is shown below (Table 1) for illustration, according to some implementations.

TABLE 1

| Cell Index | KLRG1 | CD38 | CD27 | ... | sample | source | antigen peptide |
|---|---|---|---|---|---|---|---|
| 0 | 19 | 0 | 29 | | HD10 | Flu | GILGFVFTL |
| 1 | 10 | 4 | 59 | ... | HD10 | Flu | GILGFVFTL |
| ... | ... | ... | ... | ... | ... | ... | ... |

The TargetScape analysis output contains a cell number (“cell index”), intensities for protein markers per cell and is annotated with a sample code (“sample”), antigen source (“source”), target antigen peptide (“antigen peptide”). Although only “KLRG1”, “CD38”, “CD27” are shown as examples in the table above, typical implementations can use about 30 protein markers, per cell and sample.

In some implementations, the antigen peptide specificity and the protein marker expression of each T cell are measured by flow cytometry, mass cytometry, single cell sequencing, spatial proteomics or other methods to assess multiple protein expression at a single cell level.

### Example TCR Antigen Profiling (TAP) Databases

The second set of data sets 306 and 316 are created by screening another aliquot of the above mentioned blood sample with a complementary wet-lab technology called TAP. TAP data are obtained for 1) T cells specific for a limited number of antigens, specifically those identified in the upstream TargetScape approach, and 2) T cells with unknown specificity.

TAP is single cell sequencing-based and allows to derive four types of linked data for thousands of individual cells: (i) T cell antigen peptide specificity; (ii) phenotypic protein markers (similar to TargetScape); (iii) gene expression (i.e., RNA, instead of protein); and (iv) TCR sequences. TAP analysis output is conceptually an annotated table or dataframe that contains protein marker intensities, gene expression data, antigen target and sample identifier per cell in addition to a TCR sequence representation. Data related to the level of expression of protein markers are stored similarly to the TargetScape derived marker data, and can contain information for more than 30 markers per cell, generally a superset of the TargetScape markers. Gene expression data is stored similarly, and contains roughly 36,000 RNA markers for human genes. A TCR clonotype is a group of cells that share an identical TCR sequence composition, based on CDR3A, CDR3B and V and J chain usage. An example representation of a TCR and its sequence is shown below (Table 2) for illustration. Some implementations use the concatenation of these values as clonotype identifier. TCR sequences can also be represented by their DNA sequence, which codes for a corresponding protein sequence.

TABLE 2

| | | CDR3 | | | |
|---|---|---|---|---|---|
| V Alpha | J Alpha | Alpha | V Beta | J Beta | CDR3 Beta |
| TRAV19*01 | TRAJ39*01 | CALRRLNN | TRBV6-2*01 | TRBJ1-6*01 | CASSYIQSFESS |
| | | AGNMLTF | | | YNSPLHF |

In some implementations, gene expression, protein markers and TCR sequences are linked by a common cell barcode which acts as cell identifier and are jointly stored as annotated data (e.g., using the AnnData format, described in anndata.readthedocs.io/en/latest).

In some implementations, the antigen peptide specificity, the protein marker expression, the gene expression and the TCR sequence of each T cell are measured by other single cell sequencing applications, or spatial proteomics or spatial transcriptomics, or other methods to assess multiple protein and gene expression at a single cell level.

### Overview of Data Generation

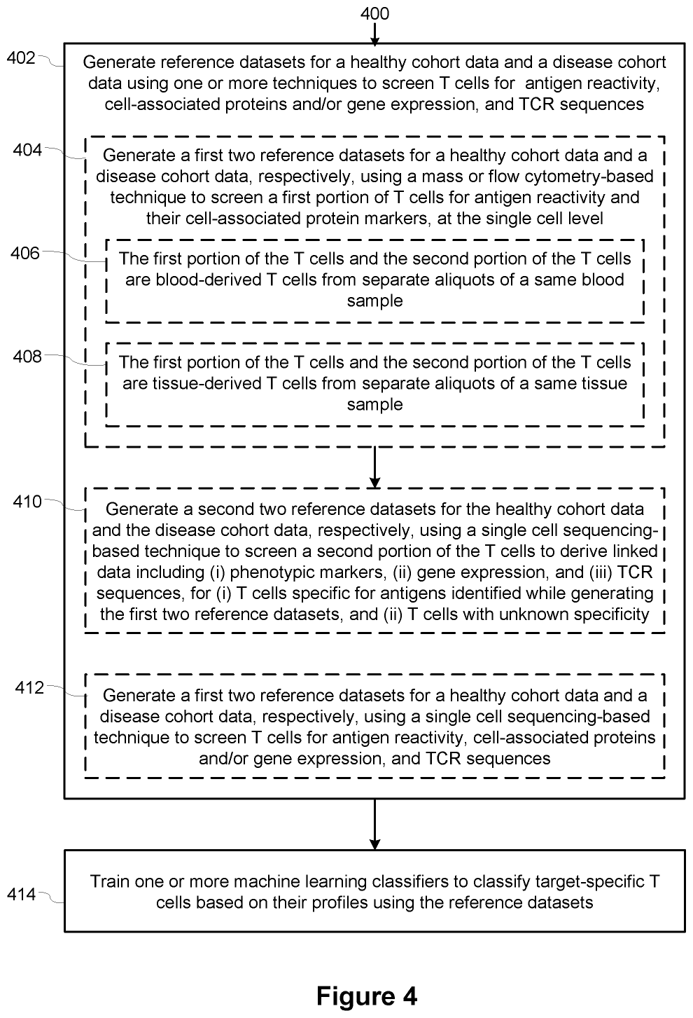

FIG. 4 shows a flowchart of an example method 400 for training a machine learning classifier for identifying target-specific T cells, according to some implementations.

The method includes generating (402) reference datasets for a healthy cohort data and a disease cohort data using one or more techniques to screen T cells for antigen reactivity, cell-associated proteins and/or gene expression, and TCR sequences. This step may include forming feature vectors by normalizing and rescaling (e.g., using log transformation and z-score conversion) T cell profiles based on the reference datasets. In some implementations, the data is organized as tables with T cells as rows and multiple measurements per cell as columns, which are then used as input for the ML methods.

In some implementations, generating the reference datasets includes generating (404) a first two reference datasets for a healthy cohort data and a disease cohort data, respectively, using a mass or flow cytometry-based technique to screen a first portion of T cells for antigen reactivity and their cell-associated protein markers, at the single cell level. Some implementations use flow cytometry if flow cytometry expands the number of parameters that can be read. Conventional mass cytometry stains for T cell profile (cell-associated marker expression), so some implementations use an improved technique that also screens for T cell antigen reactivity together with T cell profile. This is performed using a method that allows multiplexing up to several hundred different peptide-MHC multimers (tetramers) by giving each different peptide-MHC an unique three metal barcode. At the same time, some implementations stain the cells with a panel of antibodies against the cell-associated markers (phenotype). Some implementations screen all the T cells in a sample for their reactivities to all those candidate antigens, and at the same time get phenotype information of any antigen-specific T cell identified. In some implementations, the first portion of the T cells and the second portion of the T cells are (406) blood-derived T cells from separate aliquots of a same blood sample. T cells may include CD8 or CD4 T cells. Some implementations assess longitudinal blood samples from the same cancer patient. In some implementations, the first portion of the T cells and the second portion of the T cells are (408) tissue-derived T cells from separate aliquots of a same tissue sample. In some implementations, the antigens are viral antigens, tumor antigens or self-antigens.

In some implementations, generating the reference datasets further includes generating (410) a second two reference datasets for the healthy cohort data and the disease cohort data, respectively, using a single cell sequencing-based technique to screen a second portion of the T cells to derive linked data including (i) antigen specificity, (ii) phenotypic markers, (iii) (optionally) gene expression, and (iv) TCR sequences, for (i) T cells specific for antigens identified while generating the first two reference datasets, and (ii) T cells with unknown specificity. Example single cell sequencing based techniques include CITESeq, RNA sequencing and TCR sequencing.

In some implementations, generating the reference datasets includes generating (412) a first two reference datasets for a healthy cohort data and a disease cohort data, respectively, using a single cell sequencing-based technique to screen T cells for antigen reactivity, cell-associated proteins and/or gene expression, and TCR sequences. The reference datasets may be generated using specialized laboratory instruments.

Some implementations test many antigens directly with TAP or an equivalent single cell sequencing-based approach. With this, some implementations skip the TargetScape approach described in the previous section entirely.

The method also includes training (414) one or more machine learning classifiers to classify target-specific T cells based on their profiles using the reference datasets. Some implementations aggregate classification results over clonotypes, i.e. a group of cells with identical TCR composition. This step may be performed on an electronic device having one or more processors and memory. The memory stores one or more programs configured for execution by the one or more processors.

### Example Computational Approach

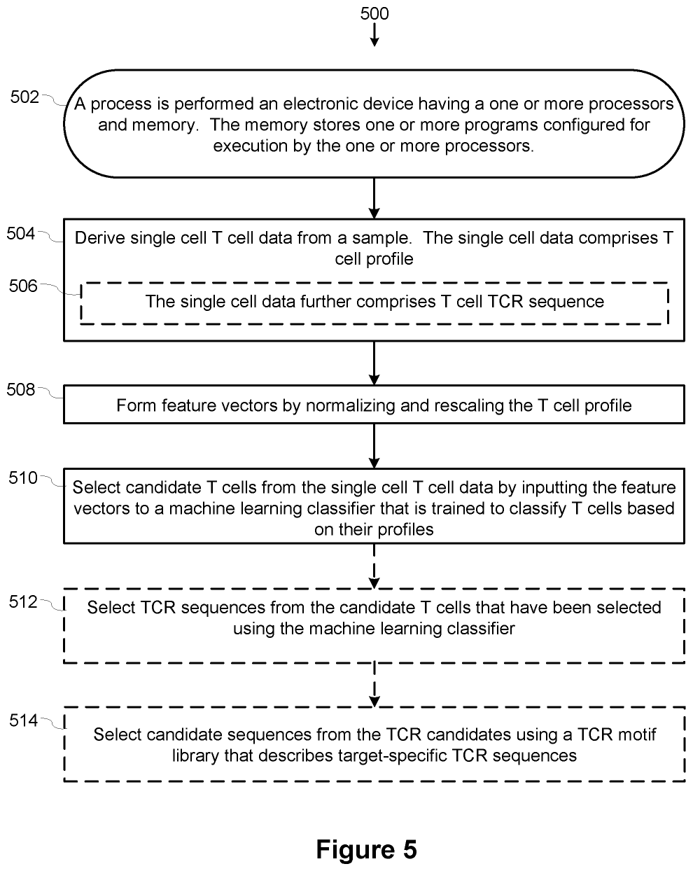

FIG. 5 shows a flowchart of an example method (500) for identifying target-specific T cells, according to some implementations. The method is performed (502) at an electronic device having one or more processors and memory. The memory stores one or more programs configured for execution by the one or more processors. The method includes deriving (504) single cell T cell data from a sample; referred to as T cell profile. A T cell profile may include cell-associated protein marker expression. A T cell profile may also include gene expression, gender, HLA types and age. In some implementations, deriving single cell data includes deriving cell-associated protein marker profiles by performing one or more of the group consisting of: mass cytometry, flow cytometry, single cell sequencing, and spatial proteomics. In some implementations, deriving single cell data includes deriving gene expression profiles by performing one or more of the group consisting of: single cell sequencing, spatial transcriptomics.

The method also includes forming (508) feature vectors by normalizing and rescaling (e.g., using log transformation and z-score conversion) the T cell profile. The method also includes selecting (510) candidate T cells from the single cell T cell data by inputting the feature vectors to a machine learning classifier that is trained to classify T cells based on their profiles.

In some implementations, the single cell data further includes (506) T cell TCR sequence, and the method further includes extracting T cell-associated (512) TCR sequences from the candidate T cells that have been selected using the machine learning classifier

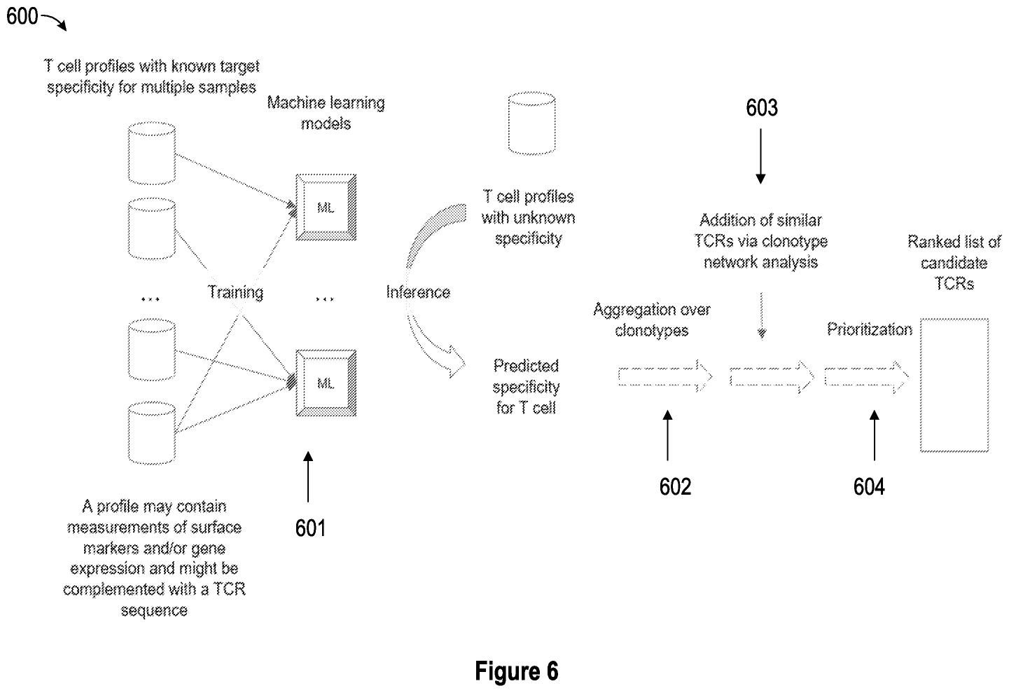

FIG. 6 is a schematic overview of the machine learning process. Input are T cell profiles that can be generated with a variety of methods and contain protein markers and/or gene expression values and/or TCR sequences (so called model features) and are, where possible, labeled with experimentally determined target specificity (e.g. antigen, virus, cancer type etc.), so-called class labels. During a training phase, the machine learning models learn generalizable signatures from the T cell profiles representative of a class.

Several different machine learning models can be trained on these so-called features using any combinations of samples, e.g. only healthy samples, only samples from a specific type of cancer etc. or a mix thereof (601). When presented with a new T cell profile of possibly unknown target specificity, the models can predict its target specificity, i.e. class label. The target specificity at the peptide, protein (e.g. pp65, LMP-2, M1, spike, PRAME, MAGE-A4), protein group (e.g. latent proteins, cancer-testis antigens, neoantigens) or target source/organism (e.g. CMV, EBV, influenza virus, SARS-COV2, tumor) resolution, is used as the class label to be predicted.

Machine learning predictions of target specificity are averaged over all cells of the same clonotype, i.e. cells with highly similar TCR sequences (602). With this clonotype-based prediction, errors for single cells can be averaged out.

In some implementations, clonotypes with similar TCR sequences are determined through clonotype network analysis, where TCR with similar CDR3A and/or CDR3B sequences are grouped together. This analysis requires the definition of a distance metric between two TCR sequences, which can either be based on sequence-only or on an encoding or physicochemical features. Sufficiently similar TCRs should bind the same epitope and thus TCRs found in this way are added to the list of candidate TCRs (603).

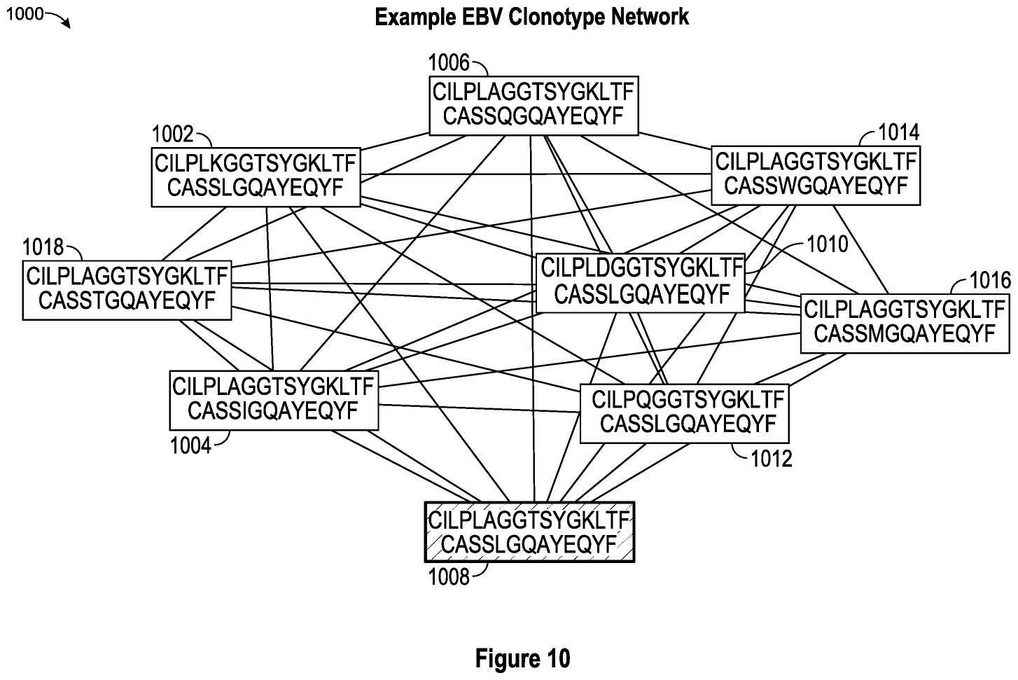

FIG. 10 shows an example clonotype network 1000 based on sequence similarity. Each vertex represents a different clonotype. Edges connect clonotypes that share a certain degree of similarity. The example shows vertices 1002, 1004, 1006, 1008, 1010, 1012, 1014, 1016, and 1018. Here, similarities/distances between CDR3 amino acid sequences were computed with TCRdist3 (Mayer-Blackwell et al., 2021), which uses a sequence-based distance metric. A distance threshold of 25 was used to derive the network. In the given example, all clonotypes were predicted to be EBV-specific by the example random forest ensemble classifier described above. One clonotype (the vertex 1008) is listed in the public database VDJdb (Bagaev et al., 2019) as EBV specific, which validates the ML predicted target specificity with an external source of truth. The TCR sequences in this network share a common CDR3 alpha and beta motif with one variable position in their alpha sequence and one variable position in their beta sequence: CILPL[ADKQ]GGTSYGKLTF and CASS[ILMQW]GQAYEQYF (brackets indicate a group of interchangeable amino acids). Sequences in this network are added to the list of candidate TCRs (see 603, FIG. 6).

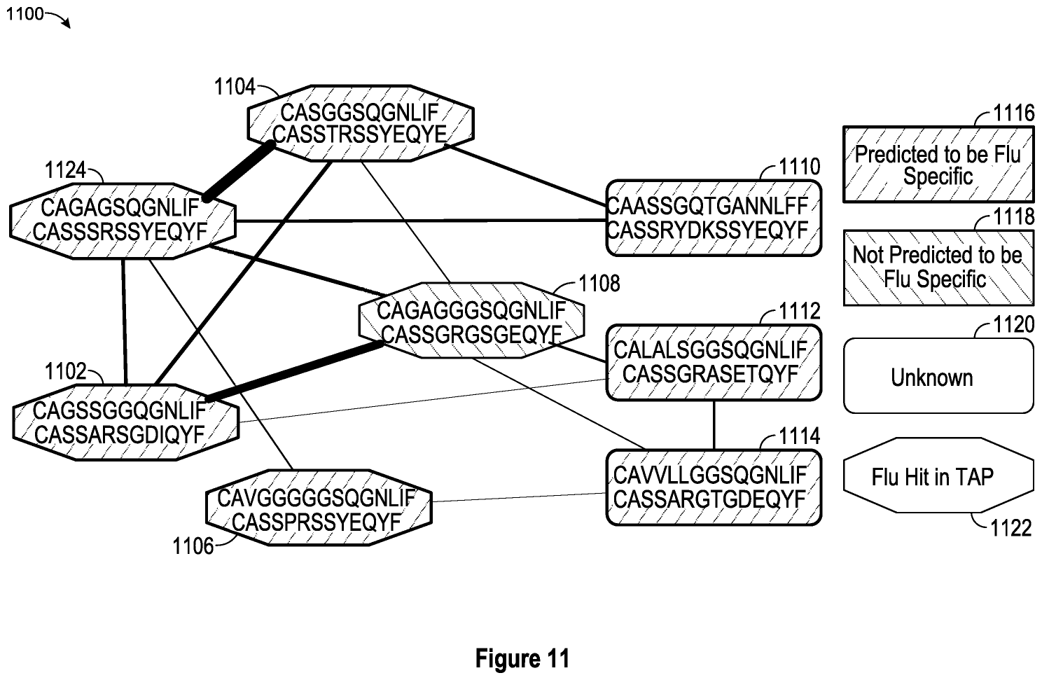

In some implementations, distance measures are calculated from TCR sequences annotated with amino acid physicochemical properties. Physical, chemical, and structural properties of the constituent amino acids of the CDR3A and CDR3B protein sequence are first encoded, by globally describing the composition, transition and distribution of the properties as described above. A global similarity metric can then be calculated for TCR sequences represented by these descriptors calculated from the physicochemical properties, using pairwise distance/similarity measures, such as Pearson or Spearman correlation or Euclidean distance. FIG. 11 shows an example network 1100 based on the physicochemical properties of the CDR3A and CDR3B TCRs. As in FIG. 10, each node or vertex represents a different clonotype. The example shows nodes 1104, 1106, 1108, 1110, 1112, 1114, and 1124. In this example, the scores are converted to a global similarity measure using the pairwise Spearman correlation for both CDR3A and CDR3B TCRs, respectively. A joined correlation matrix is computed as the mean of the CDR3A and CDR3B correlations obtained for each clonotype. The network is generated on the joint correlation matrix; two clonotypes are connected by an edge if the mean Spearman correlation between the two is above or equal to a user-defined threshold (here 0.95). In FIG. 11, edge thickness is scaled by the relative correlation coefficient to give a higher visual weight to those pairs, with a correlation coefficient closer to 1.0 and less to those that are close to the user-defined threshold. In FIG. 11, nodes 1124 and 1104 are connected by a thicker edge than the nodes 1106 and 1114, for example. Node labels show the CDR3A and CDR3B sequence of the selected clonotypes. Node shape indicates whether the respective clonotype was detected as a Flu hit in TAP (unknowns shown by shape 1120 and Flu hit shown by shape 1122) and node pattern indicates whether the respective clonotype was predicted by an ML model to be Flu specific (pattern 1116) or not (pattern 1118). In this example, all TAP hits were for the same peptide (GILGFVFTL). Hence, TCR sequences in this network become candidate sequences that are already deorphanized, i.e. they are assigned to the most likely binding antigen/peptide.

In some implementations, the list of TCR goes through a prioritization process (604). For example clonotypes/TCRs which are predicted by multiple machine learning models will receive high priority. The same applies to clonotypes/TCRs with high clonality where clonotypes/TCRs with high number of cells or high diversity in their corresponding DNA sequence are upweighted. Disease-specific clonotypes may be down-weighted if also found to be present in healthy samples or if they have a comparatively high generation probability, which can be computed with OLGA (Sethna et al., 2019).

### Example Protein Marker Derived Machine Learning Classifiers

Some implementations first train machine learning models on the TargetScape datasets 308 (also described above), such that the models can classify T cells based on their protein markers. The protein markers become, with or without further feature selection or engineering, after normalization and rescaling (e.g., using log transformation and z-score conversion), the feature vector input for the classifier (e.g., a multiclass logistic regression model or an ensemble binary random forest classifier). Some implementations may include one or more additional features such as gender, age, HLA type or disease status. The classifiers predict antigen peptide or alternatively the antigen source which is the class to be predicted. Some implementations train classifiers for specific disease antigens and their class (i.e., disease) on the disease cohort. Some implementations also train classifiers for T cells targeting peptides from common viruses, such as Influenza, CMV etc. on the healthy cohort. Some implementations train on both healthy and disease cohorts without distinguishing between them.

These classifier models are then applied to TAP datasets 310 that contain overlapping same protein markers (in the form of antigen derived tags also known as feature barcodes). The TAP-derived protein markers are normalized and rescaled (e.g. using log transformation and z-score conversion) before applying the TargetScape-derived classifier. Some implementations include additional features, such as gender, age and HLA type.

### Example Results: Protein Marker Derived Machine Learning Classifiers: Binary Random Forest Ensemble Classifiers

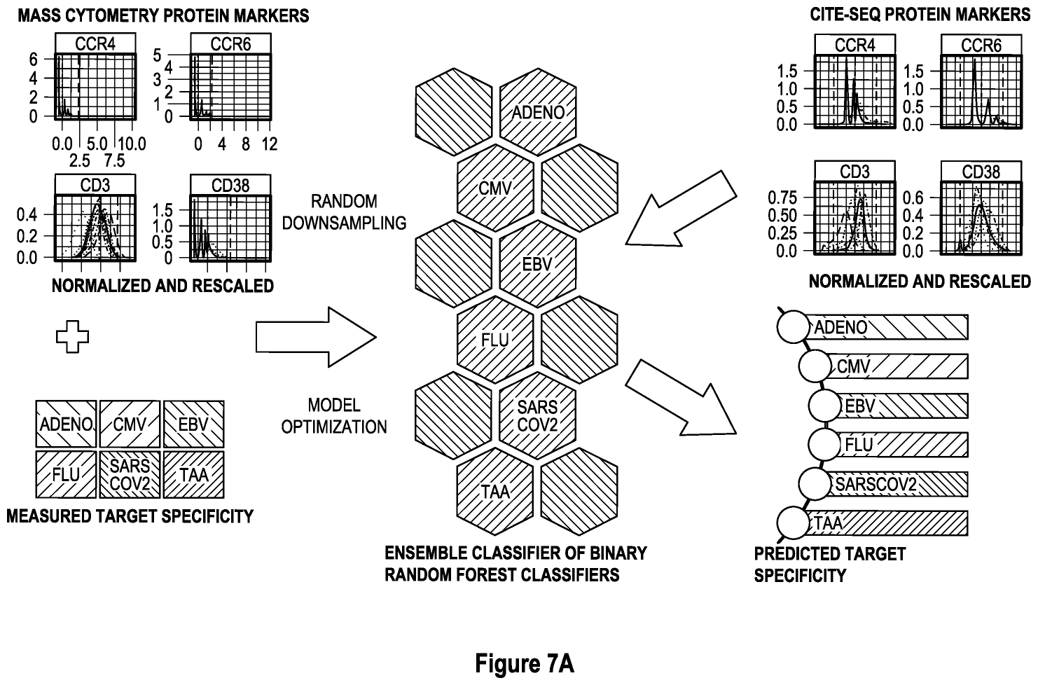

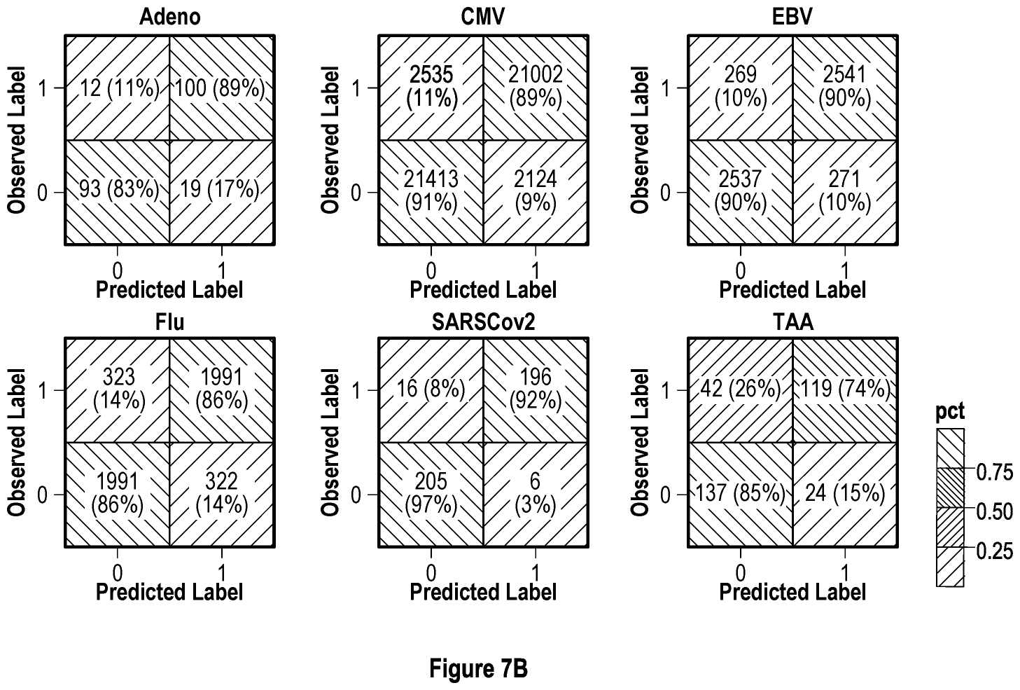

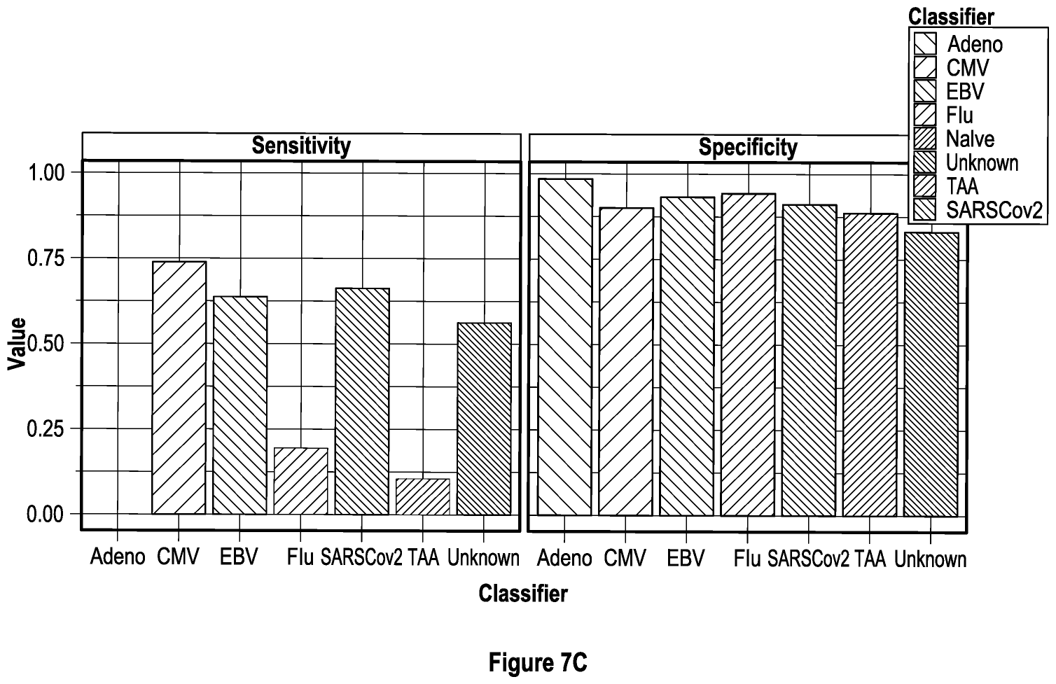

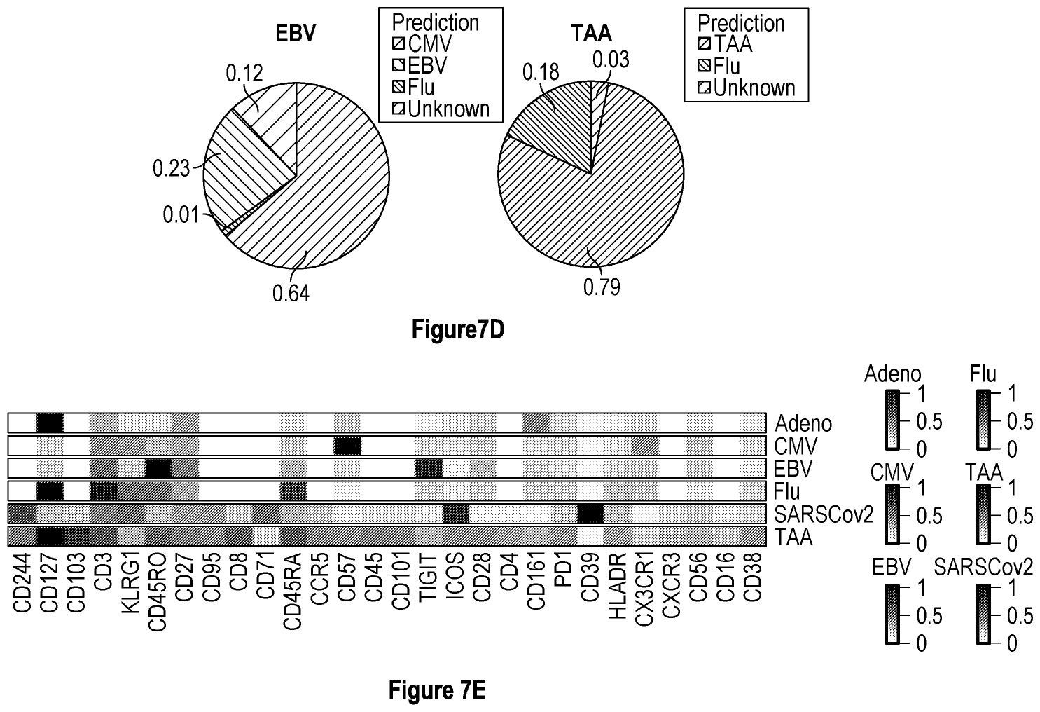

An ensemble of six random forest binary classifiers (one per virus and one for tumor associated antigens) was trained using TargetScape data of measured protein markers, normalized and re-scaled as features, as shown in FIG. 7A. Antigen measurements specific to one virus or cancer were pooled in one class (e.g. Influenza, CMV, EBV). For each binary classification specific to a virus or cancer, the positive samples were defined as the cells specific to the virus, whereas the negative samples comprise all other cells with other or unknown specificities. 10-fold cross-validation was performed for each binary classification where the dataset was randomly partitioned into 10 subsets. In each of the 10 iterations, 9 subsets (90%) were used as training and 1 subset (10%) was used for validation, where this was repeated 10 times. To account for data imbalance where there are more negative samples than positive samples, random down sampling to randomly select the negative samples to match the number of positive samples in the dataset was performed 100 times during the 10-fold cross-validation process. Parameter optimization was also applied to select the best set of parameters for the classification models. For the prediction, a threshold of 50% was applied on the class prediction probability for a positive class prediction, below which a negative class prediction label was assigned. FIG. 7B shows the observed and predicted classification labels from the testing using TargetScape data in a confusion matrix for six binary classification models trained on TargetScape data from healthy individuals. The positive class is marked as 1, while the negative class is 0, with the observed class on the y-axis and predicted class on the x-axis. The top row represents the true positive class while the bottom row represents the true negative class, whereas the first column represents the predicted negative class and the second column represents the predicted positive class. The numbers for each of the true positive, false positive, false negative and true negative are shown, with percentages calculated for each row. In summary, at least 95-99% of the target specific cells can be accurately predicted by the ensemble of binary classification models.

### Example Results: Identification of Target-Specific T Cells in TAP Data Using TargetScape-Trained Protein Marker-Based Classifiers

Target specificities of T cells were predicted on TAP data from healthy donors using the above described ensemble of six random forest binary classifiers trained on TargetScape data, as shown in FIG. 7A. Cell-associated protein marker expression from single cell T cell data of the same markers used in the TargetScape ensemble classifier were used as feature vectors after normalization and rescaling. In the ensemble classification, class probabilities are predicted by each of the six binary classifiers. The final predicted class label is assigned based on the highest class probability of the six classifiers with a threshold of 50%, otherwise labeled as cells with unknown specificities. FIG. 7C shows the sensitivity and specificity of the T-cell target-specificity predictions of TAP data using the ensemble classifier trained on TargetScape data. While the test sensitivities in correctly predicting true positives for Flu and TAA are low, the ensemble classifier is highly specific. This demonstrates that machine learning classifiers can be trained on TargetScape data and applied to TAP data, all after corresponding normalization and rescaling. The relatively low sensitivity can be counteracted by aggregating results over all cells constituting a clonotype (see next section).

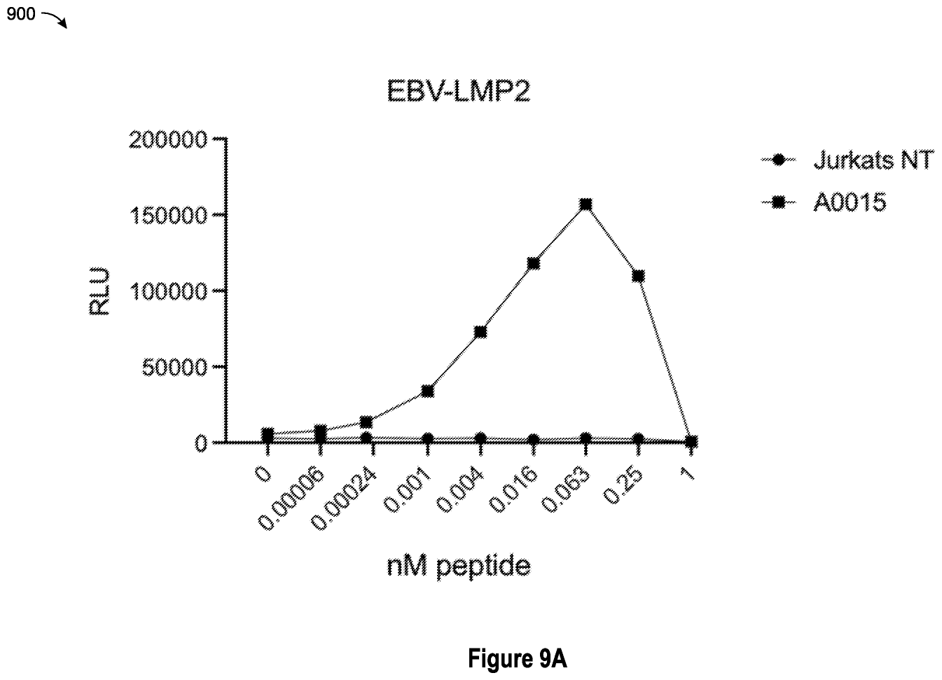

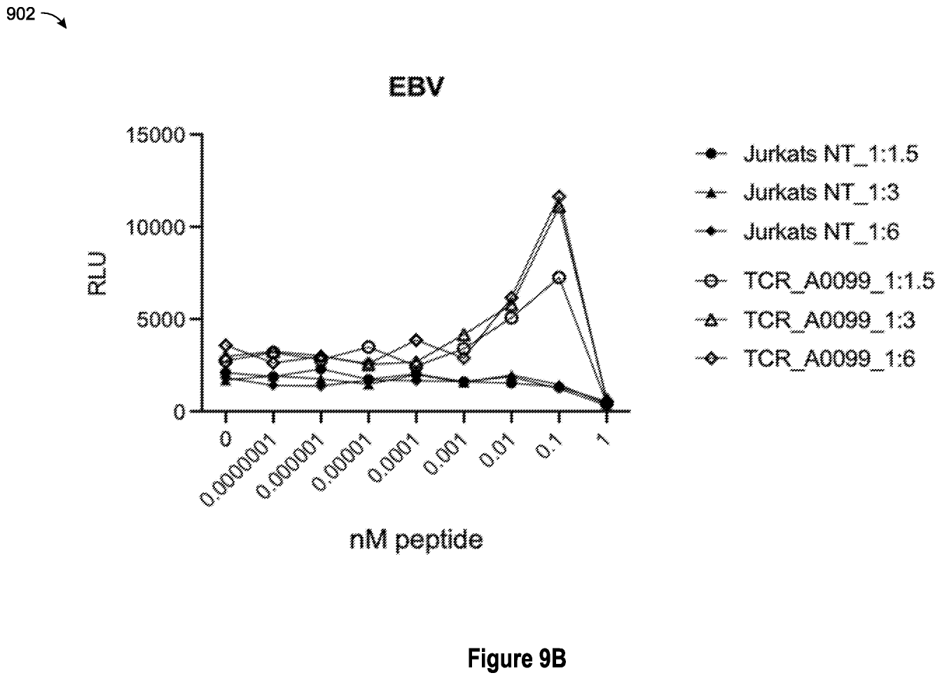

Target-specificity predictions are then aggregated over T cells with highly similar TCR sequences (clonotypes) to select candidate T cells and their TCR sequences. FIG. 7D shows two clonotype examples of aggregated T-cell target-specificity predictions (one viral and one cancer) where cells in the clonotype with different T cell profiles might have different target-specificity predictions. After aggregating, the target specificity forming the majority is used as the target specificity for this clonotype. This averages out prediction errors and prediction uncertainty. This EBV clonotype or its TCR was experimentally validated to be functional (see FIG. 9A), as was another clonotype/TCR, which was predicted to be EBV specific (FIG. 9B).

Example Results: Target-Specific T Cells Signature Derived from TargetScape-Trained Protein Marker-Based Classifiers

T cell signatures can be derived from machine learning classifiers learned from TargetScape data to distinguish between a target-specific positive class, for example a virus-specific positive class, versus others. From the random forest binary classifiers described above, the importance of each feature used in building the model to distinguish between positive and negative classes can be extracted and interpreted as T cell signature unique to the virus or cancer compared to others. Shown in FIG. 7E, protein markers are assigned weights between 0 to 1 indicating the importance of the marker for each of the binary random forest classifiers. Weights increase from lighter to darker shades. For example CD45RO is an important protein marker for the EBV class.

Example Gene Expression and/or Protein Marker-Derived Machine Learning Classifiers

The TAP databases (310) contain linked T cell-associated protein markers, gene expression and TCR data as well as antigen specificity data for some T cells. Some implementations train machine learning classifiers to predict antigen specificity from TAP derived protein markers, or gene expression or an integration of both. Gene expression and protein markers may go through feature selection and are then used as features after normalization and rescaling (e.g., using log transformation and z-score conversion). The target specificity at the peptide, protein (e.g. pp65. LMP-2, M1, spike, PRAME, MAGE-A4), protein group (e.g. latent proteins, cancer-testis antigens, neoantigens) or target source/organism (e.g. CMV, EBV, influenza virus, SARS-COV2, tumor) resolution, is used as the class label to be predicted.

Some implementations test many antigens directly with the TAP or an equivalent single cell sequencing-based approach. With this, some implementations skip the TargetScape approach described in the previous section entirely.

Example Results for Gene-Expression and/or Protein Marker-Derived Machine Learning Classifiers: Regularized Multinomial Logistic Regression

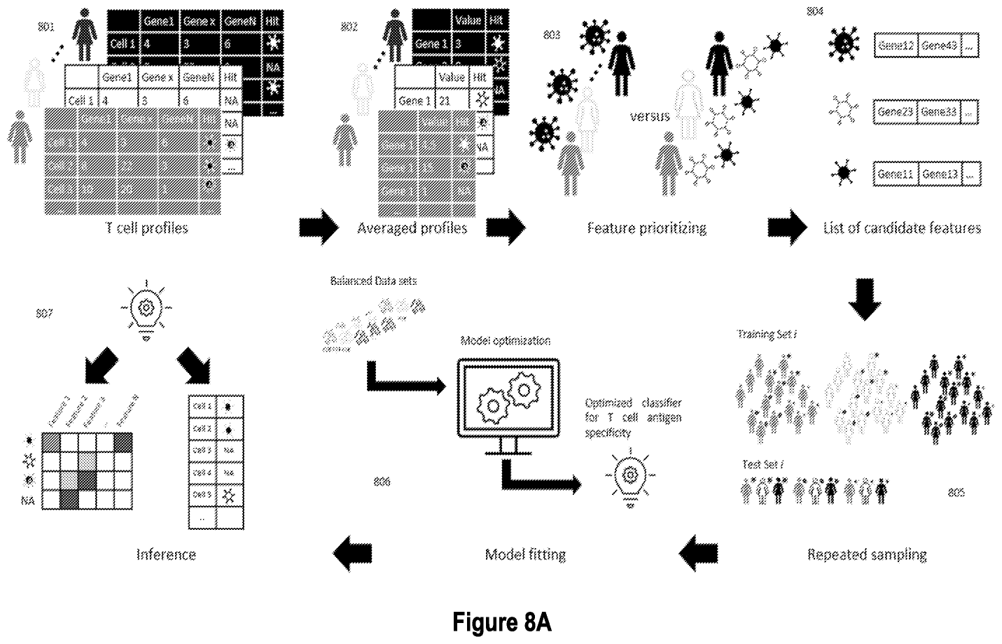

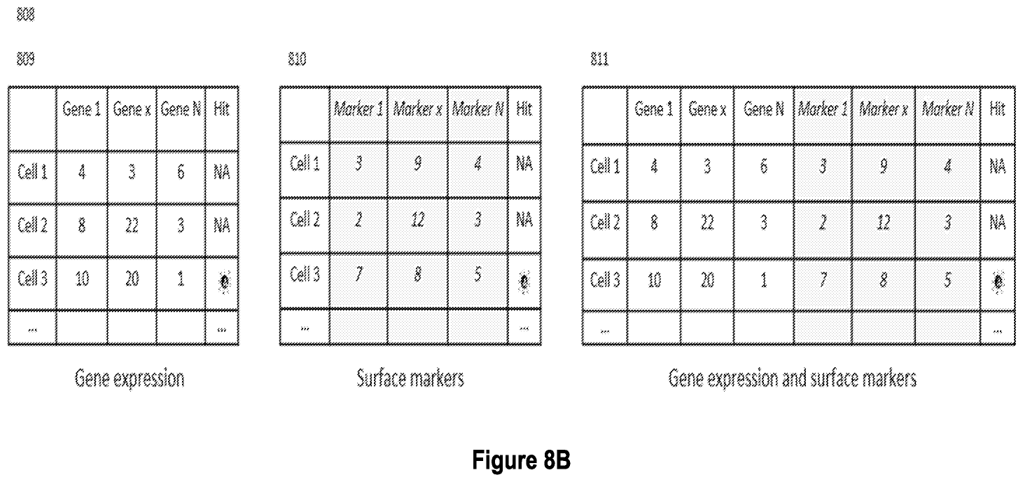

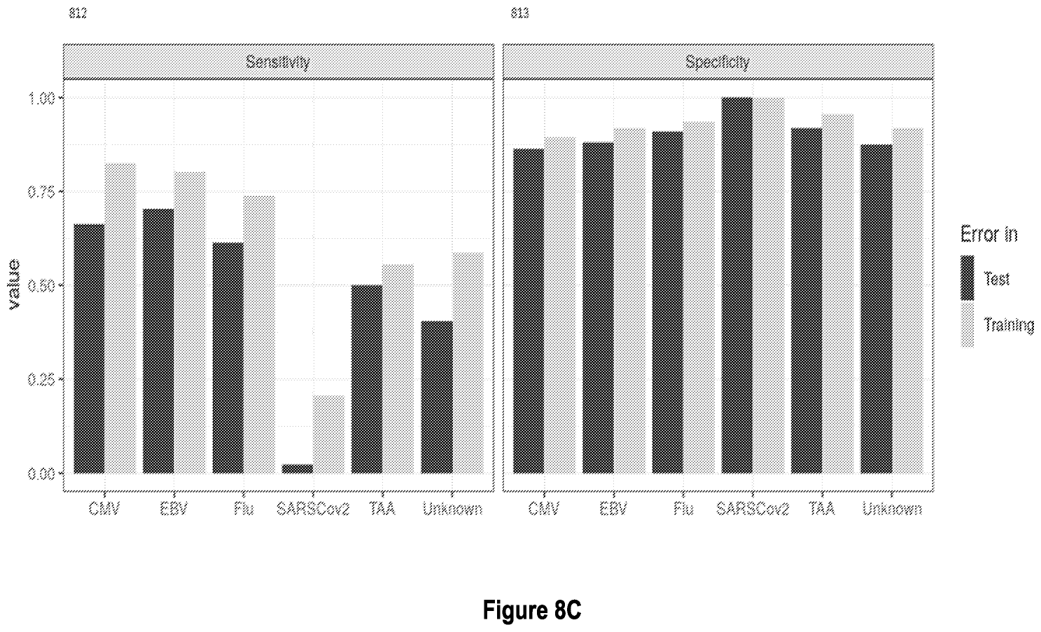

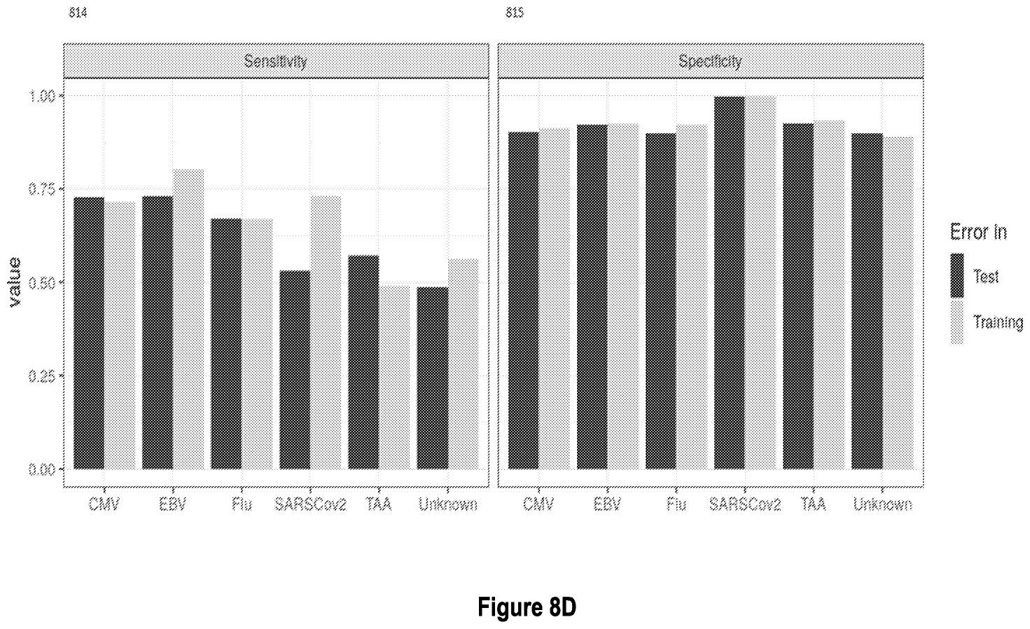

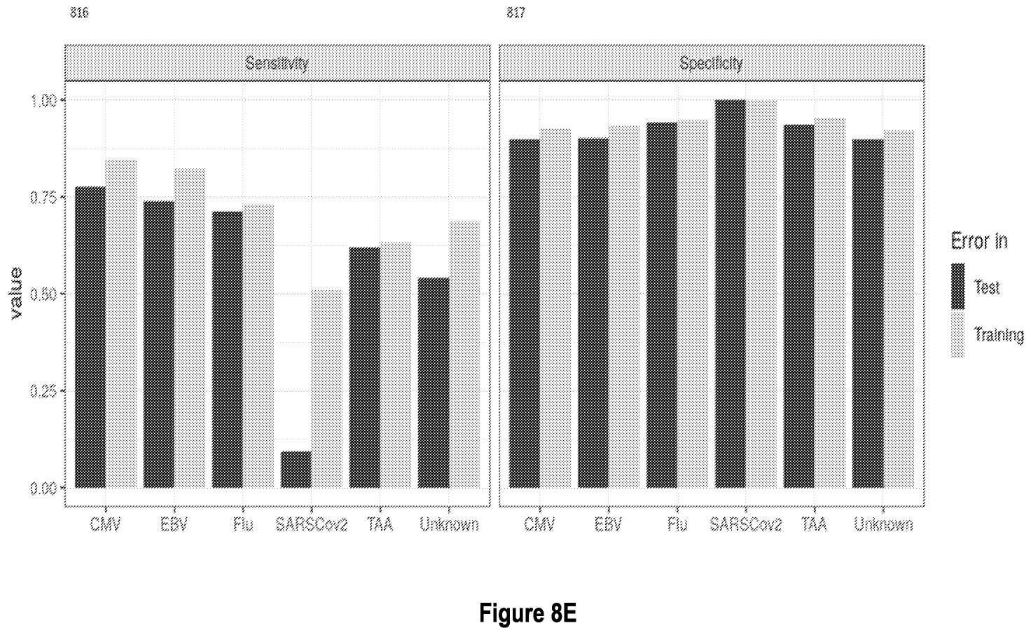

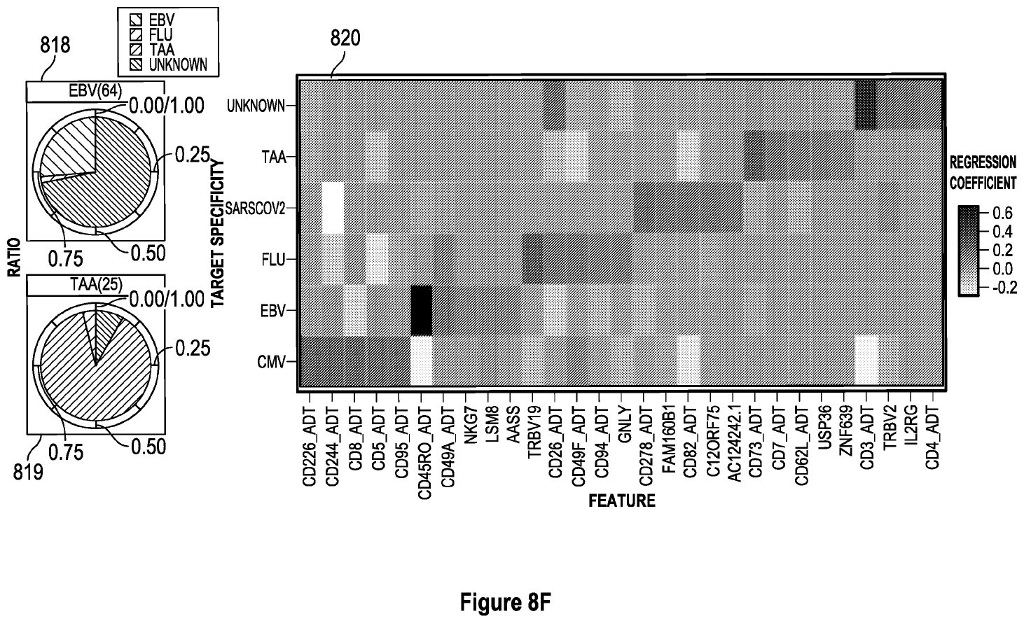

TAP data was used to train a multi-class, regularized logistic regression model on T cell data using TAP measured gene-expression markers and/or protein markers as features and using TAP measured target specificity as class to be predicted (FIG. 8A). Per individual donor, all antigen measurements are pooled in the relevant classes (e.g., Flu, CMV, EBV, TAA), yielding a donor specific profile of T cell gene expression and target specificity (801). To compute candidate marker genes prior to model learning, the T cell profiles are averaged per donor and marker, so called pseudo-bulk analysis (802). A statistical test is applied on the pseudo-bulked data to obtain a list of genes that are significantly differentially expressed between cells of different target specificity (803). By applying suitable cut-offs, this list provides a set of candidate genes which are then used as features for the multinomial logistic regression (804). To account for data imbalance, the datasets for training and testing are randomly sampled 100 times in a balanced way using a 10-fold Monte Carlo Cross (MCC) validation procedure applied to each balanced set. The MCC validation uses 80% of the balanced set for training and 20% for testing (805). The best parameter combination identified by MCC is then used for final model fitting (806). Thereby, a classifier is obtained that can be applied on previously unseen data (807).

FIG. 8B depicts various implementations of T cell profiles (808) that are used as input for the machine learning classifier (FIG. 8A). Example implementations are based on gene expression data (809), protein marker information (810), and a combination of both (811).

FIG. 8C shows performance of one of the multiclass logistic regression models trained and tested on gene expression extracted from TAP data from healthy individuals in terms of sensitivity (also known as recall) (812) and specificity (813). CMV, EBV, Flu, SARS-COV2 and TAA are labels for T cells that are specific to antigens of these classes. The “Unknown” is the catch-all label for cells for which no antigen specificity could be detected in the TAP assay; in other words, their target specificity is unknown. Across all categories, the model achieves a specificity above 80% (813).

FIG. 8D shows performance of one of the multiclass logistic regression models trained and tested on protein marker data generated by TAP data from healthy individuals in terms of sensitivity (814) and specificity (815). Across all categories, the model achieves a specificity above 80% (815), with sensitivity being at least 50% across all classes (814).

FIG. 8E shows performance of one of the multiclass logistic regression models trained and tested on both gene expression and protein marker data generated by TAP from healthy individuals in terms of sensitivity (816) and specificity (817). The instantiation using both gene expression and cell-associated protein markers achieves the highest model performance in terms of specificity (817) and also outperformed the other models in terms of sensitivity (816) for most target specificities.

FIG. 8F shows examples of clonotypes predicted to be specific for EBV (818) and TAA (819). The pie charts visualize how many cells with the same clonotype are predicted as either EBV, Flu, TAA or Unknown. The numbers behind the heading of each figure indicate the size of the clonotypes, which are 64 for EBV (818) and 25 for TAA (819), respectively. Both clonotypes are also independently predicted by the ensemble method shown in FIG. 7A (compare to FIG. 7D). The EBV clonotype (top) has been functionally validated experimentally (FIG. 9A).

FIG. 8F also includes a heatmap (820; compare also to FIG. 7E) illustrating the regression coefficients for the top five features of each class across all antigen specificity groups derived by the ML model using both gene expression and protein markers as input (811). Features labeled with the suffix “ADT” are protein markers, the remaining ones reflect gene expression estimates. A staircase pattern is clearly visible in the heatmap, suggesting that the inferred features are highly specific for the respective target specificity groups.

A selection of the most relevant genes for each class predicted by the implementation of the ML model using only gene expression data is shown in Table 3.

TABLE 3

| Multiclass | |
|---|---|
| logistic regression | Signature |
| SARSCov2 | LEPROTL1, CTLA4, AC124242.1, LYST, ANXA1 |
| TAA | LEF1, ATP5IF1, ORMDL3, FOS, LINC02446 |
| EBV | GZMK, DUSP2, HNRNPLL, PNRC1, CST7 |
| CMV | CCL5, IFITM2, CD6, GZMH, SNHG8 |
| FLU | TRBV19, GNLY, NBL1, FXYD2, KLRC1 |

### Example Application of Target-Specific T Cell Signature in Diagnosis and Immunomonitoring

T cell specificity can be used for the diagnosis of various past or present diseases. In the example shown in Table 4, T cells from blood samples of a healthy individual and of a cancer patient are analyzed by TargetScape and TAP to generate the T cell profiles. Then, the TargetScape-trained ensemble classifier and the TAP-trained multinomial logistic regression exemplified in FIGS. 7 and 8 are applied to the T cell data to derived the frequency of T cells predicted to be CMV, EBV, Flu, SARS-Cov2, and tumor (TAA)-specific. Within the healthy individual sample, only cells that show a CMV-specific signature as derived by both machine learning classifiers are detected, indicating that that individual was previously exposed to and infected with CMV. In the cancer patient sample, on the other hand, T cells with a TAA-specific signature are also detected, in addition to T cells with a CMV-specific and EBV-specific signature. Thus, the presence of the TAA-associated signature within the T cells indicates the presence of cancer in that particular individual.

TABLE 4
Frequency of CD8 T cells predicted to be viral-specific
or TAA-specific based on signature, in a sample
from a healthy donor and from a cancer patient.

| | Healthy individual | | Cancer patient | |
|---|---|---|---|---|
| | TargetScape- | TAP trained | TargetScape- | TAP trained |
| | trained | multinomial | trained | multinomial |
| Predicted | ensemble | logistic | ensemble | logistic |
| specificity | classifier | regression | classifier | regression |
| CMV | 2.9% | 2.7% | 20% | 34% |
| EBV | 0% | 0% | 1.5% | 4.2% |
| Flu | 0% | 0% | 0% | 0% |
| SARSCov2 | 0% | 0% | 0% | 0% |
| TAA | 0% | 0% | 0.3% | 0.1% |
| Unknown | 97.1% | 97.3% | 78.2% | 61.7% |

A target-specific T cell signature can also be used to monitor evolution of disease-associated target-specific T cells during disease progression or treatment. Table 5 shows individual proportions of CD8 T cells with influenza virus signature (and therefore predicted to be influenza-specific) and tumor antigen signature (and therefore predicted to be TAA-specific) in blood from two cancer patients, at two timepoints pre- and post-treatment with checkpoint blockade (T0 and T1). Checkpoint blockade treatment is expected to reinvigorate exhausted tumor-specific T cells to further proliferate and kill the tumor cells. In this example, T cells from the cancer patient blood samples were analyzed by TAP to generate the T cell profiles. Then target specificity was predicted for TAP data in samples of both timepoints using the TargetScape-trained ensemble binary random forest classifier described above. The proportions of predicted TAA-specific T cells increased post-treatment for both cancer patients while the proportions of predicted influenza-specific T cells remained relatively constant, suggesting that the checkpoint blockade treatment may be active and expanding the proportion of tumor-specific T cells in these two patients.

TABLE 5
Frequency of CD8 T cells predicted to be influenza-
specific or TAA-specific based on signature, monitored
in two cancer patients pre- and post-treatment.

| | |
|---|---|
| Patient 1 | Patient 2 |

| | T0 | T1 | T0 | T1 |
|---|---|---|---|---|
| Percent CD8 T | 2.3% | 2.2% | 3.6% | 2.8% |
| cells with | | | | |
| influenza | | | | |
| signature | | | | |
| Percent CD8 T | 12.4% | 22.6% | 10.8% | 15.7% |
| cells with TAA | | | | |
| signature | | | | |

### Example Application of Predicted Target-Specific TCR Sequence in Diagnosis and Immunomonitoring

A predicted target-specific TCR sequence (a clonotype) can also be used to monitor evolution of T cells during disease progression or treatment. Table 6 shows the change in frequency of two CD8 T cell clonotypes predicted to be TAA-specific in blood of two cancer patients, at two timepoints pre- and post-treatment with checkpoint blockade (TO and T1). Checkpoint blockade treatment is expected to reinvigorate exhausted tumor-specific T cells to further proliferate and kill the tumor cells. In this example, T cells from the cancer patient blood samples were analyzed by TAP to generate the T cell profiles. Target specificity was predicted for TAP data from samples of both timepoints using the ensemble binary random forest classifier and the multiclass logistic regression model described above. Single cell predictions were then aggregated per clonotype, i.e. T cells with the same TCR sequence. For a conservative overall prediction only expanded clonotypes (i.e., clonotypes with more than one T cell) were kept where additionally i) either model predicts TAA specificity for a clonotype in both time-points and ii) the other model also predicts either TAA or “Unknown” specificity, i.e. doesn't contradict the first model. This process resulted in identification of the two clonotypes listed in Table 6. Frequency is the number of identical predicted TAA-specific TCR sequences divided by the number of total sequenced TCR. Expansion, i.e. increase in frequency of clonotypes predicted to be TAA-specific was observed post-treatment, suggesting that the checkpoint blockade treatment may have been active in these patients and that the ML model is useful to identify and monitor tumor-specific T cells in longitudinal samples from cancer patients.

TABLE 6
Expansion of clonotypes predicted to be TAA specific pre-
and post-treatment in blood from two cancer patients.

| | | Pre-treatment T0 | | | | Post-treatment T1 | | | |
|---|---|---|---|---|---|---|---|---|---|
| | | | | ML | ML | | | ML | ML |
| Patient | Clonotype | Counts | Frequency | model 1 | model 2 | Counts | Frequency | model 1 | model 2 |
| 1 | x | 1 | 0.03% | Unknown | TAA | 11 | 0.17% | Unknown | TAA |
| 2 | y | 1 | 0.01% | TAA | TAA | 7 | 0.09% | Unknown | TAA |

Example Application of Isolated TCR Polypeptide Derived from ML Prediction.

ML-predicted TCR sequences can be expressed as isolated nucleic acids into lentiviral vectors, and the vectors can be then used to transduce host cells to express TCR polypeptides for a variety of applications. Either vector and/or host cells can be part of a pharmaceutical formulation comprising a pharmaceutically acceptable carrier. In this example, TCR (TCR_A0015) was predicted by both ensemble binary random forest classifier and multiclass logistic regression classifier (see above) to be EBV-specific (see FIGS. 7D and 8F). Details of its DNA and protein sequence can be found in Table 7. In this experiment, the TCR alpha-chain and beta-chain nucleic acid sequences were cloned and expressed into a lentiviral vector according to known methods; the lentiviral vector was then used to transduce a Jurkat luciferase reporter T cell line that does not express any endogenous TCRs. T2 target cells expressing HLA-A*02:01 were incubated with increasing concentrations of a pool of peptides derived from the LMP-2 protein of EBV and mixed with the said Jurkat T cells. Jurkat T cells specifically activated by peptide-MHC via the TCR produce luciferase. Luciferin, the substrate for luciferase, is then added along with additional reagents enabling a chemical reaction producing light. Expression of luciferase following TCR activation can thus be quantified as relative light units (RLU). An increasing response with increasing amount of peptide added to the cells is expected until reaching saturation in the system. FIG. 9A shows a graph plot 900 for the specific recognition of LMP-2-derived peptides by the transduced TCR-Jurkat T cell line, detected by luminescence signal. Non-transduced Jurkat T cells were used as negative controls and did not emit luminescence, showing therefore no specific target recognition. This example shows the functional validation of a ML-predicted TCR as being indeed specific for the predicted target. It also shows how an isolated TCR can be used to direct TCR-expressing host cells against a cell presenting the relevant peptide-MHC target.

TABLE 7
Protein and DNA sequence of TCR A0015, predicted to be EBV specific. The
CDR3 region is underlined.

| | Alpha chain | Beta chain |
|---|---|---|
| Protein | AQSVTQLDSQVPVFEEAPVELR | DAGVIQSPRHEVTEMGQEVTLRCKP |
| sequence | CNYSSSVSVYLFWYVQYPNQG | ISGHNSLFWYRQTMMRGLELLIYFN |
| | LQLLLKYLSGSTLVKGINGFEA | NNVPIDDSGMPEDRFSAKMPNASFS |
| | EFNKSQTSFHLRKPSVHISDTAE | TLKIQPSEPRDSAVYFCASSWTGNE |
| | YFCAVSALSYNQGGKLIFGQGT | QYFGPGTRLTVT |
| | ELSVKP | |
| DNA | GCCCAGTCTGTGACCCAGCTT | GATGCTGGAGTTATCCAGTCACCC |
| sequence | GACAGCCAAGTCCCTGTCTTT | CGCCATGAGGTGACAGAGATGGG |
| | GAAGAAGCCCCTGTGGAGCTG | ACAAGAAGTGACTCTGAGATGTAA |
| | AGGTGCAACTACTCATCGTCT | ACCAATTTCAGGCCACAACTCCCT |
| | GTTTCAGTGTATCTCTTCTGGT | TTTCTGGTACAGACAGACCATGAT |
| | ATGTGCAATACCCCAACCAAG | GCGGGGACTGGAGTTGCTCATTTA |
| | GACTCCAGCTTCTCCTGAAGT | CTTTAACAACAACGTTCCGATAGA |
| | ATTTATCAGGATCCACCCTGG | TGATTCAGGGATGCCCGAGGATCG |
| | TTAAAGGCATCAACGGTTTTG | ATTCTCAGCTAAGATGCCTAATGC |
| | AGGCTGAATTTAACAAGAGTC | ATCATTCTCCACTCTGAAGATCCA |
| | AAACTTCCTTCCACTTGAGGA | GCCCTCAGAACCCAGGGACTCAGC |
| | AACCCTCAGTCCATATAAGCG | TGTGTACTTCTGTGCCAGCAGCTG |
| | ACACGGCTGAGTACTTCTGTG | GACAGGGAACGAGCAGTACTTCGG |
| | CTGTGAGTGCCCTTTCTTATAA | GCCGGGCACCAGGCTCACGGTCAC |
| | CCAGGGAGGAAAGCTTATCTT | A |
| | CGGACAGGGAACGGAGTTATC | |
| | TGTGAAACCC | |

In a second example, another TCR (TCR_A0099) was predicted to be EBV specific by both ensemble binary random forest classifier and multiclass logistic regression classifier. Details of its DNA and protein sequence can be found in Table 8. In this experiment, the TCR alpha-chain and beta-chain nucleic acid sequences were cloned and expressed into a lentiviral vector according to known methods; the lentiviral vector was then used to transduce a Jurkat luciferase reporter T cell line that does not express any endogenous TCRs. PBMC target cells expressing HLA-B*35:01 (an HLA allele in common with the individual from which the TCR_A0099 was predicted) were incubated with increasing concentrations of PepTivator® EBV consensus peptide pool (Miltenyi). Jurkat T cells specifically activated by peptide-MHC via the TCR produce luciferase. Luciferin, the substrate for luciferase, is then added along with additional reagents enabling a chemical reaction producing light. Expression of luciferase following TCR activation can thus be quantified as relative light units (RLU). An increasing response with increasing amount of peptide added to the cells is expected until reaching saturation in the system. FIG. 9B shows the specific recognition of EBV peptide pool by the transduced TCR-Jurkat T cell line, measured by luminescence. TCR-Jurkat T cells were added to the PBMC target cells at different effector to target ratios: 1:1.5, 1:3, and 1:6. Non-transduced Jurkat T cells were used as negative controls at the same effector to target ratios, and did not show any specific target recognition. This example shows the functional validation of another ML-predicted TCR as being indeed specific for the predicted target. It also shows how an isolated TCR can be used to direct TCR-expressing host cells against a cell presenting the relevant peptide-MHC target.

TABLE 8
Protein and DNA sequence of TCR A0099, predicted to be EBV specific. The
CDR3 region is underlined.

| | Alpha chain | Beta chain |
|---|---|---|
| Protein | ILNVEQSPQSLHVQEGDSTNFT | EAGVAQSPRYKIIEKRQSVAFWCNPI |
| sequence | CSFPSSNFYALHWYRWETAKSP | SGHATLYWYQQILGQGPKLLIQFQN |
| | EALFVMTLNGDEKKKGRISATL | NGVVDDSQLPKDRFSAERLKGVDST |
| | NTKEGYSYLYIKGSQPEDSATY | LKIQPAKLEDSAVYLCASSSDWTAN |
| | LCAVNAGGTSYGKLTFGQGTIL | NEQFFGPGTRLTVL |
| | TVHP | |
| DNA | ATACTGAACGTGGAACAAAGT | GAAGCTGGAGTTGCCCAGTCTCCC |
| sequence | CCTCAGTCACTGCATGTTCAG | AGATATAAGATTATAGAGAAAAG |
| | GAGGGAGACAGCACCAATTTC | GCAGAGTGTGGCTTTTTGGTGCAA |
| | ACCTGCAGCTTCCCTTCCAGC | TCCTATATCTGGCCATGCTACCCTT |
| | AATTTTTATGCCTTACACTGGT | TACTGGTACCAGCAGATCCTGGGA |
| | ACAGATGGGAAACTGCAAAA | CAGGGCCCAAAGCTTCTGATTCAG |
| | AGCCCCGAGGCCTTGTTTGTA | TTTCAGAATAACGGTGTAGTGGAT |
| | ATGACTTTAAATGGGGATGAA | GATTCACAGTTGCCTAAGGATCGA |
| | AAGAAGAAAGGACGAATAAG | TTTTCTGCAGAGAGGCTCAAAGGA |
| | TGCCACTCTTAATACCAAGGA | GTAGACTCCACTCTCAAGATCCAA |
| | GGGTTACAGCTATTTGTACAT | CCTGCAAAGCTTGAGGACTCGGCC |
| | CAAAGGATCCCAGCCTGAAGA | GTGTATCTCTGTGCCAGCAGCTCC |
| | CTCAGCCACATACCTCTGTGC | GATTGGACAGCGAACAATGAGCA |
| | CGTTAATGCTGGTGGTACTAG | GTTCTTCGGGCCAGGGACACGGCT |
| | CTATGGAAAGCTGACATTTGG | CACCGTGCTA |
| | ACAAGGGACCATCTTGACTGT | |
| | CCATCCA | |

The foregoing description, for purpose of explanation, has been described with reference to specific implementations. However, the illustrative discussions above are not intended to be exhaustive or to limit the scope of the claims to the precise forms disclosed. Many modifications and variations are possible in view of the above teachings. The implementations are chosen in order to best explain the principles underlying the claims and their practical applications, to thereby enable others skilled in the art to best use the implementations with various modifications as are suited to the particular uses contemplated.

The terminology used in the description of the various described implementations herein is for the purpose of describing particular implementations only and is not intended to be limiting. As used in the description of the various described implementations and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” is, optionally, construed to mean “when” or “upon” or “in response to determining” or “in response to detecting” or “in accordance with a determination that,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event]” or “in accordance with a determination that [a stated condition or event] is detected,” depending on the context.

Some embodiments or implementations are described with respect to the following clauses:

Clause A1. A method for identifying target-specific T cells, the method comprising:
deriving single cell T cell data from a sample, wherein the data comprises T cell profiles;
forming feature vectors from the T cell profile; and
selecting candidate T cells from the single cell T cell data by inputting the feature vectors to a machine learning classifier that is trained to classify T cells based on their profiles.
   Clause A2. The method of clause A1, wherein forming feature vectors from the T cell profile includes normalizing and rescaling the T cell profile.
   Clause A3. The method of clause A1-A2, wherein the single cell data further comprises T cell TCR sequences, the method further comprising:
selecting TCR sequences from the candidate T cells that have been selected using the machine learning classifier.
   Clause A4. The method of any of clauses A1-A3, wherein selecting the candidate T cells and their TCR sequences comprises using a machine learning classifier that is trained to classify T cells based on their cell-associated protein marker profiles.
   Clause A5. The method of any of clauses A1-A3, wherein selecting the candidate T cells and their TCR sequences comprises using a machine learning classifier that is trained to classify T cells based on their gene expression profiles.
   Clause A6. The method of any of clauses A1-A3, wherein selecting the candidate T cells and their TCR sequences further comprises using a machine learning classifier that is trained to classify T cells based on integrated cell-associated protein marker and gene expression profiles.
   Clause A7. The method of any of clauses A1-A6, wherein selecting the candidate T cells and their TCR sequences further comprises aggregation of results over multiple cells with highly similar TCR sequence (clonotypes).
   Clause A8. The method of any of clauses A1-A7, wherein selecting the candidate T cells and their TCR sequences further comprises filtering for putative target-specific T cells and their TCR sequences by prioritizing candidate sequences that are for example clonally expanded, show high levels of nucleotide diversity, are common in a disease cohort data and absent in a healthy cohort etc.
   Clause A9. The method of any of clauses A1-A8, wherein selecting the candidate T cells and their TCR sequences further comprises selecting T cells and TCR with TCR sequences very similar to the sequences of the predicted TCR or to the sequences of TCR with known specificity, where such sequences may be similar based on the amino acid composition of their CDR3alpha and/or CDR3beta and/or based on physicochemical properties of their CDR3alpha and/or CDR3beta.
   Clause A10. The method of any of clauses A1-A9, wherein deriving single cell data comprises deriving cell-associated protein marker profiles by performing one or more of the group consisting of: mass cytometry, flow cytometry, single cell sequencing, and spatial proteomics.
   Clause A11. The method of any of clauses A1-A9, wherein deriving single cell data comprises deriving gene expression profiles by performing one or more of the group consisting of: single cell sequencing, spatial transcriptomics.
   Clause A12. The method of any of clauses A1-A11, further comprising:
identifying a target-specific T cell's signature by deriving cell-associated proteins and/or gene expression features and/or TCR sequence features that are common to all target-specific T cells using a machine learning classifier.
   Clause A13. The method of clause A12, further comprising:
using the signature for diagnosis of a disease comprising screening for the presence of the disease-associated target-specific T cell signature in an individual, by assessing the T cells in a blood sample for expression of cell-associated proteins and/or genes that constitute the signature, the presence of such T cells being indicative of present or past disease.
   Clause A14. The method of clause A12, further comprising:
using the signature for monitoring evolution of disease-associated target-specific T cells during disease progression or treatment, comprising i) obtaining longitudinal blood samples from individuals with a disease, or under treatment, ii) screening for the presence of the target-specific T cell signature in such longitudinal blood samples, by assessing the T cells for expression of cell-associated proteins and/or genes that constitute the signature, and iii) reporting changes in frequencies or characteristics of such target-specific T cells during time to describe the evolution of disease and/or the effect of the treatment.
   Clause A15. The method of any of clauses A1-A11, further comprising:
identifying an isolated nucleic acid or an isolated polypeptide comprising a TCR sequence, or portion thereof, based on the candidate sequences.
   Clause A16. The method of any of clauses A1-A11 or clause A15, further comprising:
using the isolated target-specific TCR, isolated nucleic acid or an isolated polypeptide comprising a TCR sequence, for diagnosis of a disease by assessing the presence of one or several target-specific TCR sequences in a blood sample or a tissue, the presence of such sequences being indicative of present or past disease.
   Clause A16. The method of any of clauses A1-A11 or clause A15, further comprising:
using the isolated target-specific TCR, isolated nucleic acid or an isolated polypeptide comprising a TCR sequence, for monitoring evolution of T cells during disease progression or treatment, comprising i) obtaining longitudinal blood or tissue samples from individuals with a disease, or under treatment, ii) screening for the presence of one or several target-specific TCR sequences in such blood or tissue samples, iii) using the target-specific TCR sequences to identify target-specific T cells, and iv) reporting changes in frequencies or characteristics of such target-specific T cells during time to describe the evolution of disease and/or the effect of the treatment.
   Clause B1. A method for training a machine learning classifier for identifying target-specific T cells, the method comprising:
generating reference datasets for healthy samples and disease samples using one or more techniques to screen T cells for antigen reactivity, cell-associated proteins and/or gene expression, and/or TCR sequences; and
training one or more machine learning classifiers to classify target-specific T cells based on their profiles using the reference datasets.
   Clause B2. The method of clause B1, wherein generating the reference datasets comprises:
generating a first two reference datasets for a healthy cohort data and a disease cohort data, respectively, using a mass or flow cytometry-based technique to screen a first portion of T cells for antigen reactivity and their cell-associated protein markers, at the single cell level.
   Clause B3. The method of clause B2, wherein generating the reference datasets further comprises:
generating a second two reference datasets for the healthy cohort data and the disease cohort data, respectively, using a single cell sequencing-based technique to screen a second portion of the T cells to derive linked data including (i) antigen reactivity, (ii) phenotypic markers, (iii) gene expression, and (iv) TCR sequences for T cells specific for antigens identified while generating the first two reference datasets, and including (i) phenotypic markers, (ii) gene expression, and (iii) TCR sequences for T cells with unknown specificity.
   Clause B4. The method of any of clauses B2-B3, wherein the first portion of the T cells and the second portion of the T cells are blood-derived T cells from separate aliquots of a same blood sample.
   Clause B5. The method of any of clauses B2-B3, wherein the first portion of the T cells and the second portion of the T cells are tissue-derived T cells from separate aliquots of a same tissue sample.
   Clause B6. The method of any of clauses B1-B5, wherein generating the reference datasets comprises:
generating a first two reference datasets for a healthy cohort data and a disease cohort data, respectively, using a single cell sequencing-based technique to screen T cells for antigen reactivity, cell-associated proteins and/or gene expression, and TCR sequences.
   Clause C1. An isolated polypeptide comprising a sequence corresponding to a TCR sequence identified by a method according to any previous claim.
   Clause C2. An expression vector comprising a nucleic acid encoding the polypeptide of clause C1.
   Clause C3. A host cell expressing a polypeptide of clause C1 encoded by a nucleic acid, wherein the polypeptide comprises a sequence corresponding to a TCR sequence identified by a method according to any previous claim.
   Clause C4. The host cell according to clause C3, wherein the host cell is a T cell.
   Clause C5. A pharmaceutical formulation comprising a vector according to clause C2, or a host cell according to any of clauses C3-C4, and a pharmaceutically acceptable carrier.

1. A method for identifying target-specific T cells, the method comprising:
deriving single cell T cell data from a sample, wherein the single cell T cell data comprises T cell profiles;
forming feature vectors from the T cell profile; and
selecting candidate T cells from the single cell T cell data by inputting the feature vectors to a machine learning classifier that is trained to classify T cells based on (i) their cell-associated protein marker profiles, (ii) their gene expression profiles, or (iii) integrated cell-associated protein marker and gene expression profiles.

2. The method of claim 1, wherein forming feature vectors from the T cell profile includes normalizing and rescaling the T cell profile.

3. The method of claim 1, wherein the single cell data further comprises T cell TCR sequences, the method further comprising:
selecting TCR sequences from the candidate T cells that have been selected using the machine learning classifier.

4. (canceled)

5. (canceled)

6. (canceled)

7. The method of claim 1, wherein selecting the candidate T cells and their TCR sequences further comprises aggregation of results over multiple cells with highly similar TCR sequence (clonotypes).

8. The method of claim 1, wherein selecting the candidate T cells and their TCR sequences further comprises filtering for putative target-specific T cells and their TCR sequences by prioritizing candidate sequences that are clonally expanded, show high levels of nucleotide diversity, are common in a disease cohort data and absent in a healthy cohort.

9. The method of claim 1, wherein selecting the candidate T cells and their TCR sequences further comprises selecting T cells and TCR with TCR sequences similar to the sequences of the predicted TCR or to the sequences of TCR with known specificity, wherein such sequences may be similar based on the amino acid composition of their CDR3alpha and/or CDR3beta and/or based on physicochemical properties of their CDR3alpha and/or CDR3beta.

10. The method of claim 1, wherein deriving single cell data comprises deriving cell-associated protein marker profiles by performing one or more of the group consisting of: mass cytometry, flow cytometry, single cell sequencing, and spatial proteomics.

11. The method of claim 1, wherein deriving single cell data comprises deriving gene expression profiles by performing one or more of the group consisting of: single cell sequencing, spatial transcriptomics.

12. A method for training a machine learning classifier for identifying target-specific T cells, the method comprising:
generating reference datasets for healthy samples and disease samples using one or more techniques to screen T cells for antigen reactivity, cell-associated proteins and/or gene expression, and/or TCR sequences; and
training one or more machine learning classifiers to classify target-specific T cells based on their profiles using the reference datasets.

13. The method of claim 12, wherein generating the reference datasets comprises:
generating a first two reference datasets for a healthy cohort data and a disease cohort data, respectively, using a mass or flow cytometry-based technique to screen a first portion of T cells for antigen reactivity and their cell-associated protein markers, at the single cell level.

14. The method of claim 13, wherein generating the reference datasets further comprises:
generating a second two reference datasets for the healthy cohort data and the disease cohort data, respectively, using a single cell sequencing-based technique to screen a second portion of the T cells to derive linked data including (i) antigen reactivity, (ii) phenotypic markers, (iii) gene expression, and (iv) TCR sequences for T cells specific for antigens identified while generating the first two reference datasets, and including (i) phenotypic markers, (ii) gene expression, and (iii) TCR sequences for T cells with unknown specificity.

15. The method of claim 13, wherein the first portion of the T cells and the second portion of the T cells are blood-derived T cells from separate aliquots of a same blood sample.

16. The method of claim 13, wherein the first portion of the T cells and the second portion of the T cells are tissue-derived T cells from separate aliquots of a same tissue sample.

17. The method of claim 12, wherein generating the reference datasets comprises:
generating a first two reference datasets for a healthy cohort data and a disease cohort data, respectively, using a single cell sequencing-based technique to screen T cells for antigen reactivity, cell-associated proteins and/or gene expression, and TCR sequences.

18. The method of claim 1, further comprising:
identifying a target-specific T cell's signature by deriving cell-associated proteins and/or gene expression features and/or TCR sequence features that are common to all target-specific T cells using a machine learning classifier.

19. The method of claim 18, further comprising:
using the signature for diagnosis of a disease comprising screening for the presence of the disease-associated target-specific T cell signature in an individual, by assessing the T cells in a blood sample for expression of cell-associated proteins and/or genes that constitute the signature, the presence of such T cells being indicative of present or past disease.

20. The method of claim 18, further comprising:
using the signature for monitoring evolution of disease-associated target-specific T cells during disease progression or treatment, comprising i) obtaining longitudinal blood samples from individuals with a disease, or under treatment, ii) screening for the presence of the target-specific T cell signature in such longitudinal blood samples, by assessing the T cells for expression of cell-associated proteins and/or genes that constitute the signature, and iii) reporting changes in frequencies or characteristics of such target-specific T cells during time to describe the evolution of disease and/or the effect of the treatment.

21. The method of claim 1, further comprising:
identifying an isolated nucleic acid or an isolated polypeptide comprising a TCR sequence, or portion thereof, based on the candidate sequences.

22. The method of 1, further comprising:
using the isolated target-specific TCR, isolated nucleic acid or an isolated polypeptide comprising a TCR sequence, for diagnosis of a disease by assessing the presence of one or several target-specific TCR sequences in a blood sample or a tissue, the presence of such sequences being indicative of present or past disease.

23. The method of claim 1, further comprising:
using the isolated target-specific TCR, isolated nucleic acid or an isolated polypeptide comprising a TCR sequence, for monitoring evolution of T cells during disease progression or treatment, comprising i) obtaining longitudinal blood or tissue samples from individuals with a disease, or under treatment, ii) screening for the presence of one or several target-specific TCR sequences in such blood or tissue samples, iii) using the target-specific TCR sequences to identify target-specific T cells, and iv) reporting changes in frequencies or characteristics of such target-specific T cells during time to describe the evolution of disease and/or the effect of the treatment.

24-28. (canceled)
